(19) United States
(12) Patent Application Publication
(10) Pub. No.: US 2011/0028344 A1
(43) Pub. Date: Feb. 3, 2011

(54) BIOMARKERS FOR ENDOMETRIAL DISEASE

(75) Inventors: David B. Krizman, Gaithersburg, MD (US); Thomas G. Guiel, Gaithersburg, MD (US)

Correspondence Address: Perkins Coie LLP, 607 Fourteenth Street, NW, Washington, DC, 20005, US

(73) Assignee: Expression Pathology, Inc., Rockville, MD (US)

(21) Appl. No.: 12/937,222

(22) PCT Filed: Apr. 13, 2009
(86) PCT No.: PCT/US09/40399

§ 371 Date: Oct. 8, 2010

Related U.S. Application Data
(60) Provisional application No. 61/044,459, filed on Apr. 11, 2008.

(51) Int. Cl. C40B 30/04; C12Q 1/02; G01N 33/574; G01N 33/50; G01N 33/53; C07K 16/18

(52) U.S. Cl. 506/9; 435/29; 436/64; 435/40.5; 435/7.1; 435/7.92; 530/387.1

(57) ABSTRACT

This patent application discloses and describes a list of proteins that are found to be differentially expressed between normal endometrial epithelial cells and early stage cancerous endometrial epithelial cells. These proteins can be used either individually or in specific combinations in diagnostic and prognostic protein assays on various biological samples from endometrial cancer patients, or individuals suspected on having endometrial cancer. In addition, these proteins are also differentially expressed between normal endometrial epithelial cells and epithelial cells of other types of endometrial disease, and thus such diseases can be diagnosed using assays based on these proteins. The full length intact proteins can be assayed or peptides derived from these proteins can be assayed as reporters for these proteins. These proteins can also be identified as “companion diagnostic” proteins, wherein they are not only differentially expressed for use as diagnostic and prognostic indicators of endometrial cancer and other endometrial diseases, but the same proteins are also targets for therapeutic intervention of endometrial cancer and other endometrial diseases.

This application claims the benefit of U.S. Provisional Application 61/044,459 filed Apr. 11, 2008, the contents of which are hereby incorporated by reference in their entirety.

### BACKGROUND OF THE INVENTION

In the United States, cancer of the endometrium is the most common cancer of the female reproductive organs. The American Cancer Society estimates there will be 39,080 new cases of cancer of the body of the uterus (uterine corpus) diagnosed in the United States during 2007. Most of these occur in the endometrium, the lining of the uterus. The American Cancer Society also estimates that about 7,400 women in the United States will die from cancers of the uterine body during 2007. About 70% of all cases are found in women between the ages of 45 and 74, with the highest number diagnosed in the 55 to 64 age group and only 8% occurring in younger women. The chance of any women being diagnosed with this cancer during her lifetime is about one in 40. There are over 500,000 women who are survivors of this cancer.

The 5-year survival rate for endometrial cancer following appropriate treatment is 5% to 95% for stage 1, 50% for stage 2, 30% for stage 3, and less than 5% for stage 4. The mainstay of patient survival and well being for endometrial cancer is early detection. Because a very high percentage of patients survive at least 5 years if their cancer is detected early, there is a great need for a test to identify early stage endometrial cancer. Endometrial cancer is primarily a sporadic disease that results from complex gene/protein interactions and hormonal selection factors. The majority of endometrial cancers are discovered when the patient develops symptomatic bleeding which then triggers a diagnostic endometrial biopsy. Upon biopsy, 21% of endometrial adenocarcinomas at the time of initial diagnosis are already in advanced stage 2-4 disease; however, if detected earlier many of these patients could achieve surgical cure by hysterectomy alone.

There are currently no routine molecular tests of practical utility for the detection of early stage endometrial cancer. In terms of early screening approaches, endometrial biopsies and curettings are too invasive to be considered screening tools. Biopsies are only used for patients who present with symptomatic bleeding. A less invasive screening method and one that is routine is the PAP smear, which is primarily intended for detection of cervical disease. However, on occasion, a cytopathologist will recognize malignant endometrial cells in the specimen. While this approach can detect endometrial cancer in its earliest stages, a PAP smear is not recommended as a sensitive means of endometrial cancer detection. Transvaginal ultrasound technology has been evaluated as a possible screening/diagnostic tool for endometrial carcinoma but this approach lacks the sensitivity and specificity needed for routine use.

The field of proteomics strives to establish the identities, quantities, structures, and biochemical and cellular functions of all proteins in an organism. Application of proteomics has proceeded mostly on a one-protein-at-a-time basis. The human proteome contains hundreds of thousands of proteins, and using recently developed proteomic techniques, changes in proteins that are overexpressed in cells within solid tissue as well as proteins that are shed into body fluids throughout disease progression can now be examined. Specific proteins, and patterns of proteins, that are found to be differentially expressed in diseased cells vs. normal cells can be reflective and diagnostic of a given disease state.

In recent years, advanced technologies and methodologies have been developed that provide an interface between clinical medicine/pathology and proteomics. High throughput global proteomic analysis technologies such as liquid-chromatography-tandem mass spectroscopy (LC-MS/MS) can be used to generate proteomic profiles from biological samples which are specific for disease. Such global profiles can be performed in all types of biological samples including frozen and fixed tissue and bodily fluids.

Without targeted, convenient, and reliable screening/diagnostic tests for cancer, the lack of molecular diagnostic assays will continue to plague the health care system and complicate efforts to detect and treat malignancies in their earliest stages. Endometrial cancer protein markers that are differentially expressed in cancerous endometrial tissue vs. normal endometrial tissue would form the foundation of a “personalized medicine” approach to reducing the suffering of women from endometrial cancer by greatly improving diagnosis of endometrial cancer, provide for improved prognostic capabilities, and provide targets for development of drugs that can more effectively treat endometrial cancer. In addition, the presence of these biomarkers in bodily fluids that result from localized shedding into the uterine lumen, and ultimately through the cervix into the vagina, or systemically into blood would present a readily accessible body fluid that can be sampled for proteomics-based screening and early detection. The development of a proteomics-based diagnostic/screening test and treatment strategies for early stage endometrial cancer would represent a significant medical advance for a “personalized medicine” approach to endometrial cancer diagnosis, prognosis, and therapy.

### SUMMARY OF THE INVENTION

In one embodiment the present invention provides methods of diagnosing the presence of endometrial disease in a human patient. The methods utilize a sample of endometrial tissue, endometrial cells, or a bodily fluid containing proteins from the patient's endometrium. In the methods the presence and level of expression of one or more of the proteins of Table 1 or Table 2 are detected in the sample. (Tables 1 and 2 are attached hereto and are incorporated herein in their entirety.) The level of expression of the detected proteins is compared to the level of expression of the same proteins in normal endometrial tissue. The differential expression of the one or more proteins indicates the presence of endometrial disease in the patient. In some embodiments the differential expression of the two or more proteins, three or more, or multiple proteins, indicates the presence of endometrial disease in the patient. In one embodiment, the disease is endometrial cancer.

In another embodiment, the invention provides methods of determining the prognosis for a human patient with endometrial disease. Such prognostic methods utilize a sample of endometrial tissue, endometrial cells, or a bodily fluid containing proteins from the patient's endometrium. The presence and level of expression of one or more of the proteins of Table 1 or Table 2 are detected in the sample. The level of expression of the detected proteins is compared to the level of expression of the same proteins in normal endometrial tissue. The differential expression of the one or more proteins indicates the expected course of disease progression in the patient. In one embodiment, the disease is endometrial cancer.

In another embodiment, the invention further provides a method of obtaining biomarkers for endometrial disease. The presence and level of expression of one or more, two or more, three or more, or four or more proteins in human endometrial epithelial tissue from a person with endometrial disease are compared to the presence and level of expression of those proteins in endometrial epithelial tissue from a person without endometrial disease; and those proteins that are either present in the diseased tissue and absent in the normal tissues or are differentially expressed in the diseased tissue compared to the normal tissue are identified. In one embodiment, the proteins are detected by mass spectroscopy, and the level of expression of the proteins is determined by spectral count quantization after said mass spectroscopy. In another embodiment, the proteins are detected and their levels of expression are determined by a protein microarray or by an immunoassay. In one embodiment, the disease is endometrial cancer.

In another embodiment, the invention provides a method of identifying protein targets for therapeutic intervention in endometrial disease. The presence and level of expression of proteins in human endometrial epithelial tissue from a person with endometrial disease are compared to the presence and level of expression of proteins in endometrial epithelial tissue from a person without endometrial disease; and those proteins that are either present in the diseased tissue and absent in the normal tissues or are differentially expressed in the diseased tissue compared to the normal tissue are identified. The identified proteins are targets for therapeutic intervention in endometrial disease. In one embodiment, the disease is endometrial cancer.

In any method of the invention, the level(s) of protein expression in samples from subjects suspected of having endometrial disease (e.g., cancer) can be determined concurrently with the level(s) of protein expression in reference or normal tissues. Alternatively, the levels of protein expression in samples from subjects suspected of having endometrial disease may be compared to the level(s) of expression of one or more proteins previously determined in normal tissue. Thus, the level of expression of one or more proteins in normal endometrial tissue employed in any detection, comparison, determination, or evaluation can be a level of expression determined prior to any detection, determination, or evaluation of the level of expression of one or more proteins (e.g., the proteins of Table 1 or Table 2) in a sample from a human patient. In one embodiment, the level of expression for any protein from a normal tissue sample is the mean level of expression observed in normal samples (e.g., all normal samples analyzed). In another embodiment, the level of expression for any protein from a normal tissue sample is the median value for the level of expression observed in normal samples.

Another embodiment of the invention further provides a collection of biomarkers for diagnosing the presence of endometrial disease in a human patient comprising one or more of the proteins of Table 1 or Table 2, or a fragment or fragments thereof. Fragments of proteins of Table 1 or Table 2 may be polypeptides comprising at least 10, 12, 15, 18, 20, 22, 25, 28 or 30 amino acid residues of a protein in Table 1 or Table 2. In one embodiment, the disease being diagnosed is endometrial cancer.

Another embodiment of the invention also provides a kit for the detection of endometrial disease in a human patient. The kit contains antibodies to one or more of the proteins of Table 1 or Table 2 or antibodies to one or more peptides derived by fragmentation of these proteins. In one embodiment, the disease is endometrial cancer.

In one embodiment the invention provides a method of diagnosing the presence of endometrial disease. In such methods a sample obtained from a human patient can be endometrial tissue, endometrial cells, or a bodily fluid containing RNA from said patient's endometrium. The presence and level of RNA encoding one or more, two or more, three or more, or four or more of the proteins of Table 1, Table 2, or the group consisting of GSTP-1 (glutathione S-transferase P), Transgelin-2, 6PGD (6 phosphogluconate dehydrogenase), and Vinculin in the sample are detected. Alterations in the level of RNA encoding any of the one or more, two or more, three or more, or four or more proteins in the sample compared to the level of RNA encoding any of those proteins in normal endometrial tissue indicates the presence of endometrial disease in the patient.

In another embodiment the invention further provides a method of determining the prognosis for a human patient with endometrial disease. In prognostic determinations a sample obtained from a human patient can be a sample of endometrial tissue, endometrial cells, or a bodily fluid containing RNA from said patient's endometrium. The presence and level of RNA encoding one or more, two or more, three or more, or four or more of the proteins of Table 1, Table 2, or the group consisting of GSTP-1, Transgelin-2, 6PGD, and Vinculin in the sample are detected. Alterations in the level of RNA encoding any of the one or more, two or more, three or more, or four or more proteins in the sample compared to the level of RNA encoding any of those proteins in normal endometrial tissue indicates the expected course of disease progression in the patient.

In one embodiment the invention provides a composition comprising two or more nuclei acid sequences, said sequences encoding all or part of a protein of Table 1 or a protein of Table 2 or complements of said sequences encoding all or part of a protein of Table 1 or a protein of Table 2. In another embodiment, the composition of nucleic acids comprise nucleic acids encoding all o part of GSTP-1, Transgelin-2, 6PGD, and Vinculin or complements of nucleic acids encoding all or part of GSTP-1, Transgelin-2, 6PGD, and Vinculin. The length of nucleic acids in such compositions may be greater than 18, 19, 20, 22, 24, 26, 28, 30, 32 or 34 nucleotides in length.

Another embodiment of the invention provides a composition for the treatment of endometrial disease in a human patient. The composition includes one or more antibodies or antibody fragments having binding affinity to one or more of the proteins of Table 1 or to one or more fragments of said one or more proteins of Table 1.

In another embodiment of the invention a composition for the treatment of endometrial disease in a human patient is provided. The composition includes one or more antibodies or antibody fragments having binding affinity to one or more of the proteins of Table 2 or to one or more fragments of said one or more proteins of Table 2.

In other embodiments, the invention also provides compositions for the treatment of endometrial disease in a human patient. The compositions include one or more antibodies or antibody fragments having binding affinity to one or more of the proteins, or fragments thereof, from group consisting of GSTP-1, Transgelin-2, 6PGD, and Vinculin, or one or more antibodies or antibody fragments having binding affinity to one or more protein fragments of the group consisting of GSTP-1, Transgelin-2, 6PGD, and Vinculin.

### DETAILED DESCRIPTION OF THE INVENTION

### Biomarkers

Methodologies at the interface between clinical medicine/pathology and proteomics were utilized to identify differentially expressed proteins between normal endometrial epithelial cells and early stage cancerous epithelial cells. The list of proteins of this invention was determined by global LC-MS/MS (liquid chromatography—tandem mass spectroscopy) profiling of cells from normal endometrial tissue and cells from cancerous endometrial tissue, and comparing those proteins that were heavily and consistently overexpressed in cancerous endometrial cells as compared to normal endometrial cells. Of note is that many or all of these proteins may be readily assayed in bodily fluids that derive from endometrial cells, such as vaginal fluid or fluids derived from blood such as plasma and serum. It is either endometrial-derived tissue, endometrial cells, or bodily fluids that would be assayed for diagnostic detection of endometrial cancer by assaying for specific protein expression from the list described herein. Also, one or more of the same proteins form the basis for a targeted therapeutic approach whereby a drug would be directed towards the proteins.

Identification of these proteins provides for the ability to detect early stage endometrial cancer in any type of biological sample collected from a subject, including fixed and frozen tissue, endometrial cells, and bodily fluid samples derived from both blood and vaginal fluids. The diagnostic and prognostic endpoint for disease analysis may not be a single analyte, but a proteomic pattern that is composed of many individual proteins, each of which individually cannot differentiate diseased from healthy individuals. This invention provides for either individual proteins, patterns of proteins, or collections of multiple proteins to be utilized for diagnosis, prognosis, and therapy of endometrial cancer and other endometrial disease, such as endometriosis and hyperplasia.

The present invention makes possible evaluation of, and treatment strategies for, endometrial cancer and other endometrial diseases in a subject. The method is useful for evaluating the presence, absence, nature, and/or extent of endometrial disease, and the specific drug target for effective therapy of the endometrial cancer and other endometrial diseases. By measuring one or more of the proteins from the list of the 140 proteins in Table 1, endometrial cancer can be diagnosed in a subject, the prognosis of that subject can be determined, and the specific drug for that subjects disease can be chosen. A sample of tissue, such as that which is surgically procured or biopsied from a subject and frozen or chemically fixed, endometrial cells, or a bodily fluid, such as blood, serum, plasma, or vaginal secretions, is examined to evaluate and measure protein expression.

Observed differences between proteins from the list of Table 1 in a biological sample from a subject with endometrial disease vs. a biological sample from a subject not having an endometrial disease represents a disease protein profile and is indicative of the presence, absence, nature, or extent of the endometrial pathology in the patient.

In one embodiment, the difference between the cancer protein profile and the reference normal protein profile comprises a difference in the amount of at least one biomarker protein from the list. The method for evaluating endometrial pathology in a subject includes discriminating between different disease states or between a disease state and normal state. Such a profile is also used to determine prognosis, which aims to monitor the extent and expectations of the progression or regression of endometrial disease in a given subject. To this end, the endometrial disease protein profile can be derived from a biological sample previously obtained from the subject, for example, a biological sample obtained prior to treatment or as part of a general health screening. The method is also well-suited to evaluate the efficacy of treatment decisions, such as drugs or surgeries. In the case of choice of drug therapy, one or more of the proteins within the endometrial disease protein profile can serve as a target for drug treatment where the drug specifically interacts with individual and specific proteins from the list of proteins.

Biological samples can be obtained from subjects in a variety of ways. For example, tissue samples may be all or a portion of a tissue sample obtained during biopsy or a surgical procedure. In some embodiments, methods for the analysis of tissues or cells or cells may utilize microdissection of tissue. In one embodiment the microdissection is conducted using laser-based techniques, such as those employed in the DIRECTOR® laser-based microdissection of tissue on slides (Expression Pathology Inc., Rockville, Md.). The slides employed in the DIRECTOR® technique employ Laser Induced Forward Transfer (LIFT), a non-contact microdissection technology utilizing a thin energy transfer coating, that permits microdissection and collection of materials from thin section samples.

The method further comprises a classification model or algorithm, based on one or more protein differences from the protein list of Table 1. In such a method, the model or algorithm assesses the differences between the test protein profile of a biological sample from a subject suspected of having endometrial disease and the reference normal protein profile from a biological sample from a subject not having an endometrial disease. In some embodiments the test protein profile would comprise two or more proteins. In other embodiments the test protein profile would comprise three or more, or four or more, or six or more, or eight or more, or ten or more of the proteins listed in Table 1.

The endometrial disease protein profile is generated using mass spectrometric methods and instruments including ion trap instruments and triple quadrupole instruments. Generally for analysis by mass spectrometry, full length intact proteins are reduced to individual peptides by treatment of protein samples with a proteolytic enzyme, such as trypsin, thus converting a complex protein sample preparation into a protein lysate consisting of peptides. Such peptide lysates are the preferred form of sample for analysis of proteins from a biological sample by mass spectrometry, where the quantitative presence of specific and individual peptides is indicative of the quantitative presence of the full length intact proteins from which the peptides derive. Analysis of all peptides simultaneously in a global fashion can be conducted on an ion trap mass spectrometry instrument. Analysis of peptides that specifically focus assays on individual and specific peptides, and thus proteins, can be performed on a triple quadrupole mass spectrometry instrument. Either or both of these types of instruments can be used to generate a protein profile to investigate the possibility of endometrial cancer or other endometrial diseases in a subject from which a biological sample was obtained.

In one embodiment, the analysis utilizes Selected Reaction Monitoring (SRM), which specifically analyzes only a single analyte, in this case a single peptide, in a complex protein mixture. SRM can advantageously quantify the absolute amount of a specific known peptide that resides in a complex mixture. SRM assays are further described in Kirkpatrick, et al., “The Absolute Quantification Strategy: a General Procedure for the Quantification of Proteins and Post-Translational Modifications,” Methods 35 (2005) 265-273, which is incorporated herein by reference in its entirety.

In another embodiment, analyses are conducted utilizing Multiple Reaction Monitoring (MRM), which performs many (e.g., more than one) SRM assays in one mass spectrometry analysis. Utilizing SRM it is possible to quantify the absolute amount of multiple specific known peptides within a complex peptide mixture. SRM assays are further described in Anderson, et al., “Quantitative Mass Spectrometric Multiple Reaction Monitoring Assays for Major Plasma Proteins,” Molecular & Cellular Proteomics, 5:573588 (2006), which is incorporated herein by reference in its entirety.

SMR and MRM assays are routinely carried out on a triple quadrupole mass spectrometer. Mass spectrometric assays of peptides may be conducted using Multiplexed Isobaric Tagging Technology such as the iTRAQ® isobaric tagging. Alternatively, mass spectrometric analysis may employ non-isobaric peptide labeling (e.g., duplexed non-isobaric peptide labeling) such as that employing mTRAQ® reagents and techniques.

Proteins and/or protein fragments (e.g., peptides) can also be assayed and quantitated using antibody-based immunological methods. For such methods, monospecific antisera or one or more monoclonal antibodies having a binding affinity to a protein of interest or a portion thereof (e.g., proteins of Table 1 and Table 2) can be produced. The antibodies can be labeled for direct or indirect detection of protein of interest. Labeling methods include, but are not limited to, iodination, biotinylation, enzyme labeling, fluorochrome attachment, and labeling by biosynthesis. Where an unlabeled primary antibody/antisera against a protein or protein fragment is employed, a labeled secondary antibody that recognizes the primary antibody/antisera may be employed in assays for detection and quanitation of the protein or protein fragment. Methods for antibody production, purification, and labeling are generally described in Harlow et al., “Antibodies: A Laboratory Manual,” Cold Spring Harbor Laboratory, pp. 53-281, 319-358 (1988)).

One assay method includes immobilizing the proteins and/or peptides from the proteins, on a microarray prior to detecting the proteins using antibody-based methods. The proteins or peptides may be obtained from whole samples or from a portion of a sample that is dissected or microdissected (see, e.g., U.S. Pat. No. 7,381,440) from a sample, such as frozen section or a formalin fixed section (e.g., a sample of section biopsy tissues) using any suitable method including detergent (e.g., sodium dodecylsulfate, SDS) solubilization.

Immobilized microarrays may be formed in a variety of formats, including, but not limited to, arrays formed on membranes (e.g., dot blots) having affinity for proteins or peptides (e.g., nitrocellulose or polyvinlyidinedifloride (PVDF)), or in microliter plates (e.g., 96 or 384 well format). Whether the microarray is formed on a membrane or in a microliter plate, the proteins of interest will be immobilized to locations in the array either by directly binding to the substrate forming the array or by binding to a substance, e.g., an antibody, with affinity for one or more of the proteins or peptides where the substance is directly or indirectly fixed to the substrate forming the array. The proteins can then be detected with antibodies specific for the protein using standard immunochemical techniques such as those for Western blotting or enzyme linked immunosorbent assay (ELISA) assay. Exemplary immunochemical assays are disclosed in Harlow et al., “Antibodies: A Laboratory Manual,” Cold Spring Harbor Laboratory, pp. 471-510 (1988).

In some embodiments, the substance having affinity for the proteins or peptides can be a monospecific antiserum or a monoclonal antibody that is bound to the substrate of the array either directly, or indirectly, such as through immobilized streptavidin. In one embodiment, the substance(s) with affinity for the protein(s) or peptide(s) of interest will be one or more antibodies immobilized (e.g., coated) on to one or more locations/wells of a microtiter plate. In another embodiment, it will be one or more antibodies immobilized in one or more locations/wells of a microtiter plate coated with streptavdin such as a Reacti-Bind™ Streptavidin Coated 384-Well Plates (Pierce, Rockford, Ill.). Where a substance having affinity for a protein or peptide is used to immobilize the protein(s) or peptide(s) of interest to a location in an array, the protein(s) or peptide(s) can be detected using a labeled antibody, or an unlabeled antibody and labeled secondary antibody, that does not interfere with immobilization of the protein(s) or peptides.

Any suitable method of detecting the presence of the immobilized protein(s) or peptide may be employed, including those methods commonly used in Western or ELISA assays. Where detection is accomplished by a labeled antibody or secondary antibody, any label that can be detected in microarray format employed can be employed. Examples of label types include enzymes, fluorescent substances, and radioisotopes. Enzymes employed as labels include, for example, alkaline phosphatase, peroxidase, glucose oxidase, tyrosinase, acid phosphatase, and the like. Where enzymes are employed as labels, any suitable substrate for the enzyme known in the art can be used for detection. For example, when the enzyme used is alkaline phosphatase, a luminescent substrate or a colorimetric substrate may be used. Examples of chemiluminescent substrates include CDP-Star® (Applied Biosystems), and ECL (Pierce, Rockford Ill.). Colorimetric substrates include, for example, p-nitrophenyl phosphate, 5-bromo-4-chloro-3-indolyl-phosphoric acid (BCIP), 4-nitro blue tetrazolium chloride (NBT), and iodotetrazolium (INT). Fluorescent substance include, for example, fluorescein isothiocyanate (FITC), green fluorescent protein (GFP), luciferin etc. Radioisotope labels include, for example, ¹²⁵I, ¹⁴C, ³²P, and ³⁵S.

Other assay methods for detecting proteins or fragments of proteins (e.g., peptides) include immunohistochemistry utilizing antibody-based protein detection, Such immunohistochemical methods may be conducted directly on intact thin tissue sections, where full length proteins are maintained intact within the tissue. Tissue preparation, fixation, and immunostaining methods are disclosed in Harlow et al., “Antibodies: A Laboratory Manual,” Cold Spring Harbor Laboratory, pp. 359-420 (1988). Another assay method includes antibody-based Western blot and ELISA protein detection methods, where the protein preparations interrogated are generally full length intact proteins. The detection methods, except for Western blots of intact full length proteins, may also be used to detect individual peptides that derive from whole intact proteins, and thus these methods do not necessarily require the detection of whole intact proteins, but can involve the detection of peptides derived from the whole intact proteins.

The present invention thus provides a useful method for detecting any and all proteins from the protein list in Table 1 and fragments thereof, including polypeptides (peptides) derived from those proteins. The presence, absence, nature, or extent of an endometrial pathology indicating an endometrial disease in a patient can be evaluated in view of the expression of one or more expressed biomarker proteins from the list, and/or a derivative peptide or peptides from the same proteins.

In yet another embodiment, the invention provides a method for screening a patient or population of patients for endometrial disease. Such methods of screening comprise assaying for the presence of one or more proteins or derivative peptides associated with endometrial pathology in a sample or samples obtained from a patient or population of patients. Alterations in the level of one or more of the proteins in the sample(s) compared to their levels in normal endometrial tissue may be employed in the screening to indicated endometrial disease. In some embodiments the method for screening may comprise assaying, two or more, three or more, or four or more proteins or derivative peptides associated with endometrial pathology in a sample or samples. In some embodiments, the protein or proteins are selected from the list in Table 1. The assay can be a mass spectrometric assay, but advantageously can also be an immunoassay, such as a Western blot, enzyme linked immunosorbent assay (ELISA), or immunohistochemical methods on intact tissue sections. A plurality of proteins or derivative peptides can be analyzed, thereby increasing the predictive power of the screening assay.

In addition to employing whole antibody molecules, fragments of antibodies having affinity for the protein(s) or protein fragment(s) of interest may be employed. Fragments of antibodies can be obtained by methods known in the art. For example, Fab, Fab′, F(ab′)₂, Fv, and/or ScFv (single chain Fv) fragments can be produced from antibodies of interest. The antibodies can also be produced and/or modified through recombinant DNA technology. In various embodiments, unmodified and/or modified antibodies and combinations of antibodies that bind to a protein or combination of proteins in Table 1 and Table 2 can be used in a pharmaceutical composition for the treatment of endometrial disease. In one embodiment, a pharmaceutical composition for the treatment of endometrial disease in a human patient includes one or more antibodies, antibody fragments, or humanized antibodies having binding affinity to one or more, or two or more, or three or more of GSTP-1, Transgelin-2, 6PGD, and Vinculin. In another embodiment, a pharmaceutical composition for the treatment of endometrial disease in a human patient includes one or more antibodies, antibody fragments, or humanized antibodies having binding affinity to one or more, or two or more, or three or more of the proteins listed in Table 1.

In addition to analyzing and targeting proteins of interest, RNA encoding these proteins can also be used as markers for the diagnosis, prognosis, and treatment of endometrial disease. Where RNA (or a cDNA thereof) is used as a marker it serves as a surrogate for analysis of the presence of the proteins themselves. As with analysis based on the presence of the proteins themselves, methods of diagnosis and prognosis will be improved in their ability to identify individuals with endometrial disease, such as cancer, when more than one type of nucleic acid (e.g., mRNA) encoding more than one of the proteins of Table 1 or Table 2 is used in any assessment.

In one embodiment, RNA of interest can be detected in tissues and cells by in situ hybridization techniques known in the art. Such techniques are described in, for example, “Current Protocols in Molecular Biology,” Ausubel et al. (Eds.), see Current Protocols Publishing, Sections 14.3.1-14.3.14 (1989). In some embodiments, by comparing hybridization signal from a patient's sample to a normal sample, a changed amount and/or localization pattern of RNA encoding one or more of the proteins in Table 1 and Table 2 can indicate the presence and/or progression of endometrial disease or provide a prognosis for a patient with endometrial disease.

In another embodiment, RNA encoding a protein of interest (e.g., a protein from Table 1 or 2) can be subject to analysis as a surrogate for examining the levels of proteins in the sample. In some embodiments, total cellular RNA, cytoplasmic RNA, or poly(A)+ RNA (i.e., mRNA) can be extracted from endometrial tissue, endometrial cells, or a bodily fluid containing RNA from the patient's endometrium and subjected to other analyses. In some embodiments, assays such as RT-PCR, Northern blot, serial analysis of gene expression (SAGE), differential display PCR (DD-PCR), and representational difference analysis (RDA) can be used to qualitatively and/or quantitatively measure RNA of interest. Methods for preparing and analyzing total and poly(A)+ RNA are well known and are described generally in Sambrook et al., “Molecular Cloning-A Laboratory Manual,” 2^{nd} Edition, Cold Spring Harbor Laboratory, Vol. 1-3 (1989) and “Current Protocols in Molecular Biology,” Ausubel et al. (Eds.), Current Protocols Publishing, Vol. 2 (1994).

Microarrays or DNA-chips can also be used to measure differential gene expression. Generally, microarray measurements involve a comparison of the amount of mRNA in a patient sample against a control or reference sample (e.g., normal/healthy tissue). The amount of RNA transcripts can be measured where complementary nucleic acid probes are immobilized on the array. The nucleic acid probes can be derived from genomic or cDNA libraries, from fully sequenced clones, from partially sequenced cDNAs known as expressed sequence tags (ESTs), or synthetically made on the microarray surface or substrate. Methods for obtaining such DNA molecules are generally known in the art (see, e.g., “Current Protocols in Molecular Biology,” Ausubel et al. (Eds.), Current Protocols Publishing, Vol. 2 (1994). Alternatively, oligonucleotides may be synthesized by conventional methods, such as phosphoramidite-based synthesis. In some embodiments, the probes on a DNA microarray include sequences of genes or gene fragments encoding one or more, two or more, three or more, four or more, five or more, or 10 or more of the proteins from Table 1 or Table 2.

### Identifying the Biomarkers

The proteins of this invention were selected by their patterns of differential protein expression between normal endometrial epithelium and early stage cancerous endometrial epithelium as assayed directly in endometrial tissue obtained by surgery. Thus, the data are directly obtained from the normal and diseased cells as previously residing in normal and cancer patients.

Data were collected by the methodology of mass spectrometry of protein lysates from these tissues and cells. Protein lysates obtained from these cells contain all the necessary information about differential protein expression, and this information has been utilized to determine which proteins are expressed in which collection of cells from which tissue by mass spectrometry (MS). MS data is presented as identification of the total number of peptides in each protein lysate. Each protein lysate is turned into a collection of peptides by digestion of intact polypeptides with the protease trypsin, which is the favored format for MS analysis of proteins. Once as many peptides are identified as possible in a single MS analysis of a single lysate, then that list of peptides is compared to the list of peptides identified across all lysates in a study set. Thus, the starting point for determining differential protein expression by mass spectrometry is the list of peptides found expressed in one sample as compared to another sample, or one group of samples as compared to another group.

The way in which to classify differential protein expression from these lists of peptides is to first determine which proteins are represented by a given list of peptides, and then to count the total number of different peptides identified for each protein. Collating data in this way is called the Spectral Count method. The method is described in Old et al., “Comparison of Label-Free Methods for Quantifying Human Proteins by Shotgun Proteomics,” Molecular & Cellular Proteomics 4:1487-1502 (2005), which is incorporated herein by reference in its entirety. The spectral count for a given protein is thus based on the total number of unique and different peptides identified for that protein, which is a relative indicator for the abundance of that protein in the protein lysate that was analyzed by MS. This is a mathematical method that provides the ability to compare spectral count abundances for a given protein from one sample to the next, and between individual proteins within a given sample.

Spectral counts between thousands of individual proteins across multiple samples obtained from both normal endometrial tissue and cancerous endometrial tissue were compared. Selection of the proteins was based upon which proteins showed higher spectral count abundance in large percentages of cancerous endometrial tissue vs. normal endometrial tissue. These proteins are shown in Table 1.

Data for endometrial cancer samples appears in the entries for samples 1-33 in both Table 1 and Table 2, and the data for normal tissue samples appears in the entries for samples 34-45 in both Tables 1 and 2. Where values of “NA” appear in those tables, NA is defined as “not applicable.”

Table 1 shows protein names down the left-hand column and the tissue samples shown on the top across from left to right. The data demonstrating the differential expression pattern for each protein between cancer and normal are shown in the columns at the right side of the Table. The data shown are the total and the average number of peptides for each protein present in the cancer and normal samples. Those data are followed by a ratio which demonstrates the level of increased expression for each protein in cancer over normal. The higher the ratio, the greater each protein is expressed in early stage endometrial cancer over normal endometrium. The higher ratio for expression of a protein in cancer over normal is a direct indicator of the potential for each protein to be a marker of early stage endometrial cancer. The final columns of data show the percentage of both cancer tissue and normal tissues expressing each protein. The higher the percentage in cancer over normal is a direct indicator of the potential for each protein to be a marker of disease. Thus, each of these proteins is a biomarker of early stage endometrial cancer that can be used for diagnosis, prognosis, or therapeutic targets of endometrial cancer.

### Use of the Biomarkers

The use of the identified proteins as biomarkers could be very advantageous in efforts to improve treatment of patients with early stage endometrial cancer. The over expression of one or more proteins in endometrial cancer versus normal endometrium, and the ability to assay for this over-expression in a biological sample, can be used to determine whether or not a person suspected of having endometrial cancer either does or does not have endometrial cancer. In addition, certain patterns of expression of multiple proteins in combination may be more effective at identifying individuals with endometrial cancer than any one or two proteins individually. As an assessment of multiple proteins may be more effective and accurate in the identification of individuals with endometrial cancer, this invention includes the correlation of multiple proteins simultaneously in a single biological sample from an individual suspected of having early stage endometrial cancer as a means of assessing, diagnosing and providing a prognosis for individuals.

The early detection and treatment of endometrial cancer gives rise to a greater likelihood that treatment will be successful and, consequently, it is imperative that endometrial cancer be detected and treatment begun as early as possible. The diagnosis of the stage of cancer is an important aspect in determining the course of treatment, such as, whether or not surgery is indicated and whether or not chemotherapy should be used with or without radiation. An improved and more accurate diagnosis would provide enhanced information about the best course of treatment. Assays of one or more of the proteins from Table 1, which can serve as diagnostic biomarkers of early stage endometrial cancer, may provide enhanced information regarding the presence of early stage endometrial cancer that might otherwise go unobserved, thereby improving the detection and treatment when the chances of success are greatest.

Over-expression of one or more proteins in endometrial cancer versus normal endometrium, and the ability to assay for this over-expression in a biological sample, can be used as an aide to determine which therapeutic agent is chosen to achieve the best course of disease treatment. Where one or more, or two or more, or three or more. or four or more of the proteins identified in this invention (e.g., the proteins in Tables 1 or 2) can be targeted directly with one or more drugs or antibodies, used alone or in combination, endometrial cancer cells may be killed in preference to normal cells. The preferential effect on cancer cells arises from the increased expression of the proteins found in Tables 1 or 2 in cancer cells, relative to normal endometrial cells.

The type of biological sample assayed for one or more of these proteins as biomarkers of early stage endometrial cancer can vary. For example, it includes biopsied tissue and tissue removed during surgery. The tissue can be fresh, frozen, and/or chemically fixed such as that which is preserved in formalin and other chemical fixatives of the like. Whole blood and its components, such as serum and plasma, can also be used as samples in the assays described herein. Finally, other bodily fluids, such as vaginal secretions and secretions from the endometrium, can be assayed for expression of one or more of the proteins from Table 1.

### EXAMPLE

Thirty three (33) early stage endometrial cancers and twelve (12) normal endometrial tissues were interrogated for differential protein expression that correlates to cancer. Where proteins are found to be differentially expressed in cancer cells, detection of their presence in tissue samples may be employed to improve the diagnosis, prognosis, or therapy of endometrial cancer. All of the cancers were FIGO International Federation of Gynecology and Obstetrics grade 1 or 2, negative for metastasis or lymph node involvement, and all were moderately to well-differentiated. Normal endometrial tissues and samples were considered to be normal based upon histopathological criteria and showed no signs of endometrial cancer, and/or hyperplasia an/or endometriosis. Within the normal samples there were an equal number of proliferative and secretory stage tissues.

Thin tissue sections were prepared from each tissue for use in histological analysis and for procurement of epithelial cells from both early stage endometrial cancer and normal endometrium. Global mass spectrometry profiling of all lysates followed by spectral count quantitation indicated differential expression of proteins that can act as biomarkers of early stage endometrial cancer.

Soluble protein lysates were prepared from microdissected cancerous and normal epithelial cells obtained from each of the 45 tissue samples. For normal tissue lysates, formalin fixed paraffin embedded normal endometrial tissues from twelve subjects were employed. Similarly, endometrial cancer cells collected from thirty three subjects were employed. Approximately 30,000 cells from relevant epithelial cell regions in each tissue were procured by laser-based microdissection using Director™ microdissection slides (Expression Pathology, Inc. Gaithersburg, Md.). Microdissected cells were processed using a Liquid Tissue® MS Protein preparation kit according to the manufacturer's directions (Expression Pathology, Inc. Gaithersburg, Md.). Prior to mass spectroscopy samples were desalted using a C-18 Zip-Tip microcolumn (Millipore, Billeric, Mass.).

Each lysate consisted of the total protein content of the microdissected cells digested into predictable peptide fragments by the protease trypsin. In this form, each protein lysate can be evaluated by the technology of mass spectrometry for identification and quantification of the proteins present in each lysate. In addition, total mass spectrometry data across all samples is used to determine differential protein expression between individual samples and between normal endometrial cells and cancerous endometrial cells.

Global mass spectrometry analysis of each trypsin-digested protein lysate was performed as follows. Liquid chromatography (LC) was performed using a Dionex UltiMate® 3000 system (Dionex Corporation, Sunnyvale, Calif.) coupled on-line to a Thermo Fisher linear ion trap mass spectrometer (MS) (Thermo Fisher Scientific Inc., Waltham, Mass.). LC separation of the sample was performed using a 75 μm ID×360 μm OD×10-cm-long fused silica capillary column (Polymicro Technologies, Phoenix, Ariz.) packed with 5 μm, 300 Å pore size, Jupiter C-18 stationary phase (Phenomenex, Torrence, Calif.). Protein lysates, prepared as described aboves were concentrated and re-suspended in a suitable injection solution. After injecting 5 μl of the re-suspended protein lysate, the column was washed with 98% mobile phase A (0.1% formic acid in water) for 30 min and peptides were eluted using a linear gradient from 2% mobile phase B (0.1% formic acid in acetonitrile) to 42% mobile phase B in 140 min. At 140 min the mobile phase was changed to 98% B and the column was eluted with a 2% A and 98% B mobile phase for an additional 20 min, all at a constant flow rate of 250 mL/min. The Linear Ion Trap Mass Spectrometer (LITMS) was operated in a data-dependent MS/MS mode in which each full MS scan (precursor ion selection scan range of m/z 350-1800) was followed by seven MS/MS scans where the seven most abundant peptide molecular ions were selected for tandem MS using a relative collision-induced dissociation (CID) energy of 35%. Dynamic exclusion was utilized to minimize redundant selection of peptides for CID.

Tandem mass spectra were searched against the UniProt Homo sapiens proteome database (http://www.expasy.org) using the SEQUEST search algorithm in BioWorks software from Thermo Fisher (Thermo Fisher Scientific Inc., Waltham, Mass.). Peptides were considered legitimately identified if they achieved specific charge state and proteolytic cleavage-dependent cross-correlation (Xeorr) scores of 1.9 for [M+H]1+, 2.2 for [M+2H]2+, and 3.1 for [M+3H]3+, and a minimum delta correlation score (˜Cn) of 0.08. Additionally, the identified peptides were unique to their associated proteins, since the data analysis excluded conserved peptides common to multiple proteins (Sequest ‘count’ must be null). This level of filtering yielded list of peptides with 98% or higher identification confidence.

Using the high confidence peptide data, peptide lists for each sample were combined and redundant peptide identifications were eliminated to generate a list of unique peptides. Each peptide in the list was already associated with a protein, so that the list was easily converted to a list of proteins; specifically, a list of unique proteins was created for each patient sample. Based on these data, a quantitative analysis to determine differential protein expression between cancer and normal was performed using the Spectral Count Quantitation method. Spectral Count Quantitation is the process of counting the number of unique peptides associated with each protein. A value of 4 beside a protein name (in the accompanying tables) reflects that there were 4 unique peptides that were associated with that particular protein. Although there may have been numerous repeated identifications for any of the individual peptides, the count was based on unique peptides and not total peptides. This count directly correlates to the relative abundance of each particular protein; accordingly, the greater the number of unique peptides identified for a protein, the greater the relative expression of that protein in any particular sample.

In the overall data analysis, the number of peptides across the sample set, i.e. cancer versus normal, were summed and divided by the total number of samples in the set to generate an average peptide count. In this analysis several sets of data were developed to identify differentially expressed proteins. By way of example, if there were a total of 99 peptides identified for Protein X in the entire dataset from 33 cancer samples, then the average peptide count (re: Spectral Count) was 3 (99 divided by 33). The data analysis also examined the number of times a particular protein (Protein X) was identified by at least 7 or more peptides across all the cancer samples to generate a percentage of samples where the expression of that protein was identified by mass spectrometry. For the cancer data set, Protein X had to demonstrate at least 1 or more peptides in at least 26 or more of the 33 cancer patient samples to achieve an 80% or higher expression level.

Proteins were identified whose derived quantitative expression levels showed the presence of the protein in a greater number of cancer samples (at least about 20%, or at least about 25%, or at least about 30%, or at least about 33%, or at least about 35%, or at least about 40%) vs. normal samples. Also, proteins were identified having at least a 2 fold increase in expression in samples of cancer vs. normal tissue (or cells) as determined by a ratio of the average number of peptides identified by mass spectrometry for each protein in both the cancer samples and the normal samples. Thus, over-expression of a particular protein was determined by at least a 2 fold increase in its spectral count in at least 33% of all cancer samples as compared to normal samples in the present example. In other embodiments, over expression of a particular protein can determined by at least a 2 fold increase in its spectral count in at least about 20%, or at least about 25%, or at least about 30%, or at least about 33%, or at least about 35%, or at least about 40% of all cancer samples as compared to normal samples. These criteria were established because those proteins that are identified by greater numbers of unique peptides in endometrial cancer cells over normal endometrial cells are the most likely candidates for new biomarkers of endometrial cancer. The total cumulative data for each of the 140 proteins identified using the methods described above, across all samples, are shown in Table 1.

Table 1 summarizes the processed data where the name of each protein identified as described above is listed on the left side and the samples analyzed are listed on the top from left to right, where each sample has been numbered from 1-33 for the cancer samples and 34-45 for the normal samples. The data are based on identification of unique peptides where the number of unique peptides identified for each of these proteins listed on the left for each sample follows from left to the right. At the far right there are summary functions for the peptide data for each protein. The first summary function shows the total sum of all peptide identifications for each protein across all cancer samples. The next summary function shows the total sum of all peptide identifications for each protein across all normal samples. The following two summary functions show the average number of unique peptides for each of the proteins per cancer sample and per normal sample. The values in these two summary functions are used to develop the expression ratio between cancer and normal samples. The expression ratio is the next summary function shown where all values are either non-existent, in the case where no peptides for a given protein are identified in any normal sample, or have a value of 2 or greater, in the case where some number peptides for a given protein are identified in at least one normal sample. The value of 2 indicates at least a 2-fold increase of expression for a particular protein in endometrial cancer tissue over normal endometrial tissue. The final two summary functions show the total percentage of the 33 cancer samples and the 12 normal samples where these proteins were identified as expressed.

TABLE 1

| | Column | | | | | | | | |
|---|---|---|---|---|---|---|---|---|---|
| | | | | 4 | 5 | 6 | 7 | 8 | 9 |
| Sample# | 1 | 2 | 3 | Sample # | | | | | |
| Row | Protein Name | Reference | Accession | 1 | 2 | 3 | 4 | 5 | 6 |
| 1 | Elongation factor 2 (EF-2) - Homo sapiens (Human) | EF2_HUMAN | P13639 | 8 | 4 | 4 | 5 | 4 | 4 |
| 2 | Protein disulfide-isomerase precursor (EC 5.3.4.1) (PDI) (Prolyl 4-hydroxylase subunit beta) (Cellular thyroid | PDIA1_HUMAN | P07237 | 5 | 8 | 8 | 5 | 7 | 7 |
| | hormone-binding protein) (p55) - Homo sapiens | | | | | | | | |
| 3 | Protein disulfide-isomerase A4 precursor (EC 5.3.4.1) (Protein ERp-72) (ERp72) - Homo sapiens | PDIA4_HUMAN | P13667 | 9 | 5 | 6 | 6 | 3 | 9 |
| 4 | Glutathione S-transferase P (EC 2.5.1.18) (GST class-pi) (GSTP1-1) - Homo sapiens (Human) | GSTP1_HUMAN | P09211 | 5 | 5 | 6 | 6 | 6 | 5 |
| 5 | Prohibitin-2 (B-cell receptor-associated protein BAP37) (Repressor of estrogen receptor activity) (D-prohibitin) - Homo sapiens | PHB2_HUMAN | Q99623 | 6 | 2 | 3 | 2 | 3 | 1 |
| | (Human) | | | | | | | | |
| 6 | Glyceraldehyde-3-phosphate dehydrogenase (EC 1.2.1.12) (GAPDH) - Homo sapiens (Human) | G3P_HUMAN | P04406 | 14 | 12 | 13 | 10 | 11 | 13 |
| 7 | 14-3-3 protein zeta/delta (Protein kinase C inhibitor protein 1) (KCIP-1) - Homo sapiens (Human) | 1433Z_HUMAN | P63104 | 7 | 4 | 5 | 6 | 7 | 7 |
| 8 | Annexin A1 (Annexin I) (Lipocortin I) (Calpactin II) (Chromobindin-9) (p35) (Phospholipase A2 inhibitory | ANXA1_HUMAN | P04083 | 1 | 4 | 2 | 3 | 3 | 6 |
| | protein) - Homo sapiens (Human) | | | | | | | | |
| 9 | Peptidyl-prolyl cis-trans isomerase (EC 5.2.1.8) - Homo sapiens (Human) | Q6IBH5_HUMAN | Q6IBH5 | 5 | 4 | 5 | 5 | | 6 |
| 10 | Transgelin-2 (SM22-alpha homolog) - Homo sapiens (Human) | TAGL2_HUMAN | P37802 | 7 | 5 | 6 | 5 | 5 | 6 |
| 11 | 6-phosphogluconate dehydrogenase, decarboxylating (EC 1.1.1.44) - Homo sapiens (Human) | 6PGD_HUMAN | P52209 | 6 | 5 | 4 | 6 | 5 | 7 |
| 12 | Vinculin (Metavinculin) - Homo sapiens (Human) | VINC_HUMAN | P18206 | 11 | 17 | 5 | 11 | 11 | 21 |
| 13 | T-complex protein 1 subunit delta (TCP-1-delta) (CCT-delta) (Stimulator of TAR RNA-binding) - Homo sapiens (Human) | TCPD_HUMAN | P50991 | 2 | 2 | 5 | 2 | 5 | 6 |
| 14 | Malate dehydrogenase, mitochondrial precursor (EC 1.1.1.37) - Homo sapiens (Human) | MDHM_HUMAN | P40926 | 6 | 4 | 3 | 3 | 4 | 7 |
| 15 | ELAV-like protein 1 (Hu-antigen R) (HuR) - Homo sapiens (Human) | ELAV1_HUMAN | Q15717 | 2 | 2 | 1 | 1 | 4 | 2 |
| 16 | Collagen alpha-3(VI) chain precursor - Homo sapiens (Human) | CO6A3_HUMAN | P12111 | 5 | 6 | 8 | 10 | 13 | 20 |
| 17 | Junctophilin-1 (Junctophilin type 1) (JP-1) - Homo sapiens (Human) | JPH1_HUMAN | Q9HDC5 | | | 1 | 1 | 1 | |
| 18 | Transmembrane emp24 domain-containing protein 10 precursor (Transmembrane protein Tmp21) (21 kDa transmembrane- | TMEDA_HUMAN | P49755 | 3 | 3 | 1 | 1 | 2 | 1 |
| | trafficking protein) (p24delta) (S31III125) (S31I125) (Tmp-21-I) - Homo sapiens (Human) | | | | | | | | |
| 19 | Galectin-3-binding protein precursor (Lectin galactoside-binding soluble 3-binding protein) (Mac-2-binding | LG3BP_HUMAN | Q08380 | 3 | 4 | 6 | 3 | 1 | |
| | protein) (Mac-2 BP) (MAC2BP) (Tumor-associated antigen 90K) - Homo sapiens (Human) | | | | | | | | |
| 20 | Elongation factor 1-gamma (EF-1-gamma) (eEF-1B gamma) - Homo sapiens (Human) | EF1G_HUMAN | P26641 | 6 | 4 | 5 | 4 | 4 | 4 |
| 21 | 14-3-3 protein theta (14-3-3 protein tau) (14-3-3 protein T-cell) (HS1 protein) - Homo sapiens (Human) | 1433T_HUMAN | P27348 | 3 | 2 | 1 | 5 | 3 | 3 |
| 22 | Dynein heavy chain, cytosolic (DYHC) (Cytoplasmic dynein heavy chain 1) (DHC1) (Dynein heavy chain 1, | DYHC_HUMAN | Q14204 | 11 | 14 | 20 | 13 | 9 | 11 |
| | cytoplasmic 1) - Homo sapiens (Human) | | | | | | | | |
| 23 | Dolichyl-diphosphooligosaccharide--protein glycosyltransferase 67 kDa subunit precursor (EC 2.4.1.119) | RIB1_HUMAN | P04843 | 5 | 5 | 4 | 4 | 5 | 3 |
| | (Ribophorin I) (RPN-I) - Homo sapiens (Human) | | | | | | | | |
| 24 | UDP-glucose 6-dehydrogenase (EC 1.1.1.22) (UDP-Glc dehydrogenase) (UDP-GlcDH) (UDPGDH) - Homo | UGDH_HUMAN | O60701 | 2 | 5 | 5 | 2 | 6 | 5 |
| | sapiens (Human) | | | | | | | | |
| 25 | Clathrin heavy chain 1 (CLH-17) - Homo sapiens (Human) | CLH1_HUMAN | Q00610 | 10 | 10 | 9 | 12 | 8 | 13 |
| 26 | MARCKS-related protein (MARCKS-like protein 1) (Macrophage myristoylated alanine-rich C kinase | MRP_HUMAN | P49006 | 1 | 2 | 2 | | 1 | 2 |
| | substrate) (Mac-MARCKS) (MacMARCKS) - Homo sapiens (Human) | | | | | | | | |
| 27 | Tenascin precursor (TN) (Tenascin-C) (TN-C) (Hexabrachion) (Cytotactin) (Neuronectin) (GMEM) (JI) | TENA_HUMAN | P24821 | | 9 | 1 | 3 | 6 | 2 |
| | (Myotendinous antigen)(Glioma-associated-extracellular matrix antigen)(GP 150-225) Homo sapiens | | | | | | | | |
| 28 | UPF0027 protein C22orf28 - Homo sapiens (Human) | CV028_HUMAN | Q9Y3I0 | 4 | 3 | 4 | 3 | 4 | 4 |
| 29 | 60S ribosomal protein L27a - Homo sapiens (Human) | RL27A_HUMAN | P46776 | 3 | 2 | 2 | 1 | 1 | 3 |
| 30 | 14-3-3 protein eta (Protein AS1) - Homo sapiens (Human) | 1433F_HUMAN | Q04917 | 2 | 1 | 2 | 1 | 2 | 1 |
| 31 | Proteasome activator complex subunit 1 (Proteasome activator 28-alpha subunit) (PA28alpha) (PA28a) | PSME1_HUMAN | Q06323 | 3 | 1 | 4 | 1 | 2 | 4 |
| | (Activator of multicatalytic protease subunit 1) (11S regulator complex subunit alpha) (REG-alpha) (Interferon | | | | | | | | |
| 32 | 40S ribosomal protein S25 - Homo sapiens (Human) | RS25_HUMAN | P62851 | 1 | 1 | 1 | 1 | 1 | |
| 33 | Glucosidase 2 subunit beta precursor (Glucosidase II subunit beta) (Protein kinase C substrate, 60.1 kDa protein, heavy chain) | GLU2B_HUMAN | P14314 | 3 | 4 | 5 | 2 | 3 | 6 |
| | (PKCSH) (80K-H protein) - Homo sapiens (Human) | | | | | | | | |
| 34 | Transketolase (EC 2.2.1.1) (TK) - Homo sapiens (Human) | TKT_HUMAN | P29401 | 4 | 2 | 4 | 3 | 7 | 6 |
| 35 | 60S ribosomal protein L18 - Homo sapiens (Human) | RL18_HUMAN | Q07020 | 2 | 2 | 2 | 3 | 3 | 1 |
| 36 | Protein S100-A6 (S100 calcium-binding protein A6) (Calcyclin) (Prolactin receptor-associated protein) (PRA) (Growth factor- | S10A6_HUMAN | P06703 | | 1 | | 1 | 2 | 1 |
| | inducible protein 2A9) (MLN 4) - Homo sapiens (Human) | | | | | | | | |
| 37 | T-complex protein 1 subunit beta (TCP-1-beta) (CCT-beta) - Homo sapiens (Human) | TCPB_HUMAN | P78371 | 7 | 4 | 3 | 4 | 5 | 3 |
| 38 | Aspartyl-tRNA synthetase, cytoplasmic (EC 6.1.1.12) (Aspartate--tRNA ligase) (AspRS) (Cell proliferation- | SYDC_HUMAN | P14868 | 2 | | 1 | 1 | 1 | 4 |
| | inducing gene 40 protein) - Homo sapiens (Human) | | | | | | | | |
| 39 | Malate dehydrogenase, cytoplasmic (EC 1.1.1.37) (Cytosolic malate dehydrogenase) - Homo sapiens (Human) | MDHC_HUMAN | P40925 | 3 | 2 | 4 | 2 | | 1 |
| 40 | Protein S100-A9 (S100 calcium-binding protein A9) (Calgranulin-B) (Migration inhibitory factor-related | S10A9_HUMAN | P06702 | 2 | 5 | 2 | | 2 | 2 |
| | protein 14) (MRP-14) (P14) (Leukocyte L1 complex heavy chain) (Calprotectin L1H subunit) - Homo sapiens | | | | | | | | |
| 41 | Caldesmon (CDM) - Homo sapiens (Human) | CALD1_HUMAN | Q05682 | 3 | 7 | 2 | 10 | 8 | 10 |
| 42 | Transient receptor potential cation channel subfamily V member 3 (TrpV3) (Vanilloid receptor-like 3) (VRL-3) - Homo sapiens | TRPV3_HUMAN | Q8NET8 | | 1 | 1 | | | 1 |
| | (Human) | | | | | | | | |
| 43 | C-1-tetrahydrofolate synthase, cytoplasmic (C1-THF synthase) [Includes: Methylenetetrahydrofolate | C1TC_HUMAN | P11586 | 2 | 2 | 1 | 2 | 2 | 2 |
| | dehydrogenase (EC 1.5.1.5); Methenyltetrahydrofolate cyclohydrolase (EC 3.5.4.9); Formyltetrahydrofolate | | | | | | | | |
| 44 | 150 kDa oxygen-regulated protein precursor (Orp150) (Hypoxia up-regulated 1) - Homo sapiens (Human) | OXRP_HUMAN | Q9Y4L1 | 8 | 4 | 4 | 6 | 3 | 5 |
| 45 | Pyruvate kinase isozymes M1/M2 (EC 2.7.1.40) (Pyruvate kinase muscle isozyme) (Pyruvate kinase 2/3) | KPYM_HUMAN | P14618 | 1 | 2 | 2 | 2 | 2 | 2 |
| | (Cytosolic thyroid hormone-binding protein) (CTHBP) (THBP1) - Homo sapiens (Human) | | | | | | | | |
| 46 | DNA-(apurinic or apyrimidinic site) lyase (EC 4.2.99.18) (AP endonuclease 1) (APEX nuclease) (APEN) (REF-1 protein) - | APEX1_HUMAN | P27695 | 4 | 1 | 2 | 4 | 4 | 3 |
| | Homo sapiens (Human) | | | | | | | | |
| 47 | Mucin-5B precursor (Mucin-5 subtype B, tracheobronchial) (High molecular weight salivary mucin MG1) | MUC5B_HUMAN | Q9HC84 | | 1 | 1 | 11 | | |
| | (Sublingual gland mucin) - Homo sapiens (Human) | | | | | | | | |
| 48 | Hypothetical protein SSR4 (Fragment) - Homo sapiens (Human) | Q0JSX1_HUMA | Q0JSX1 | 1 | 1 | | 1 | | 2 |
| 49 | Vesicle-trafficking protein SEC22b (SEC22 vesicle-trafficking protein homolog B) (SEC22 vesicle-trafficking | SC22B_HUMAN | O75396 | 2 | 4 | 2 | 2 | 1 | 3 |
| | protein-like 1) (ERS24) (ERS-24) - Homo sapiens (Human) | | | | | | | | |
| 50 | Moesin (Membrane-organizing extension spike protein) - Homo sapiens (Human) | MOES_HUMAN | P26038 | | 3 | 1 | | 2 | 4 |
| 51 | Lamin-B1 - Homo sapiens (Human) | LMNB1_HUMAN | P20700 | 7 | 10 | 9 | 12 | 10 | 7 |
| 52 | Polymeric-immunoglobulin receptor precursor (Poly-Ig receptor) (PIGR) (Hepatocellular carcinoma-associated | PIGR_HUMAN | P01833 | | 6 | 15 | 20 | 2 | 1 |
| | protein TB6) [Contains: Secretory component] - Homo sapiens (Human) | | | | | | | | |
| 53 | Glucose-6-phosphate isomerase (EC 5.3.1.9) (GPI) (Phosphoglucose isomerase) (PGI) (Phospho-hexose | G6PI_HUMAN | P06744 | 6 | 7 | 4 | 7 | 4 | 7 |
| | isomerase) (PHI) (Neuroleukin) (NLK) (Sperm antigen 36) (SA-36) - Homo sapiens (Human) | | | | | | | | |
| 54 | Heterogeneous nuclear ribonucleoproteins C1/C2 (hnRNP C1/hnRNP C2) - Homo sapiens (Human) | HNRPC_HUMAN | P07910 | 4 | 4 | 1 | 4 | 3 | 2 |
| 55 | Delta 1-pyrroline-5-carboxylate synthetase (P5CS) (Aldehyde dehydrogenase 18 family member A1) [Includes: | P5CS_HUMAN | P54886 | 4 | 3 | 3 | 5 | 2 | 7 |
| | Glutamate 5-kinase (EC 2.7.2.11) (Gamma-glutamyl kinase) (GK); Gamma-glutamyl phosphate reductase | | | | | | | | |
| 65 | Matrin-3 - Homo sapiens (Human) | MATR3_HUMAN | P43243 | 3 | 4 | 5 | 2 | 3 | 3 |
| 57 | Tryptophanyl-tRNA synthetase, cytoplasmic (EC 6.1.1.2) (Tryptophan--tRNA ligase) (TrpRS) (IFP53) (hWRS) | SYWC_HUMAN | P23381 | | 1 | | | | |
| | Homo sapiens (Human) | | | | | | | | |
| 58 | Fatty acid synthase (EC 2.3.1.85) [Includes: [Acyl-carrier-protein] S-acetyltransferase (EC 2.3.1.38); [Acyl- | FAS_HUMAN | P49327 | 8 | 2 | 6 | 7 | 4 | 6 |
| | carrier-protein] S-malonyltransferase (EC 2.3.1.39); 3-oxoacyl-[acyl-carrier-protein] synthase (EC 2.3.1.41); 3- | | | | | | | | |
| 59 | Importin beta-1 subunit (Karyopherin beta-1 subunit) (Nuclear factor P97) (Importin 90) - Homo sapiens | IMB1_HUMAN | Q14974 | 3 | 3 | 2 | 4 | 2 | 3 |
| | (Human) | | | | | | | | |
| 60 | Alcohol dehydrogenase [NADP+] (EC 1.1.1.2) (Aldehyde reductase) (Aldo-keto reductase family 1 member A1) - Homo sapiens | AK1A1_HUMAN | P14550 | 3 | 4 | 3 | 3 | 1 | 4 |
| | (Human) | | | | | | | | |
| 61 | Heterogeneous nuclear ribonucleoprotein Q (hnRNP Q) (hnRNP-Q) (Synaptotagmin-binding, cytoplasmic RNA- | HNRPQ_HUMAN | O60506 | 2 | 2 | | | 2 | 2 |
| | interacting protein) (Glycine- and tyrosine-rich RNA-binding protein) (GRY-RBP) (NS1-associated protein 1) - | | | | | | | | |
| 62 | Dolichyl-diphosphooligosaccharide-protein glycosyltransferase - Homo sapiens (Human) | Q5VWA5_HUMA | Q5VWA5 | | 2 | | | | |
| 63 | Villin 2 (Ezrin) - Homo sapiens (Human) | Q4VX75_HUMA | Q4VX75 | | 1 | 1 | 1 | 1 | 1 |
| 64 | ATP-dependent DNA helicase 2 subunit 2 (EC 3.6.1.- ) (ATP-dependent DNA helicase II 80 kDa subunit) | KU86_HUMAN | P13010 | 5 | 4 | 3 | 2 | 5 | 4 |
| | (Lupus Ku autoantigen protein p86) (Ku86) (Ku80) (86 kDa subunit of Ku antigen) (Thyroid-lupus autoantigen) | | | | | | | | |
| 65 | 60S ribosomal protein L4 (L1) - Homo sapiens (Human) | RL4_HUMAN | P36578 | 2 | 3 | 4 | 2 | 5 | 3 |
| 66 | Musculin (Activated B-cell factor 1) (ABF-1) - Homo sapiens (Human) | MUSC_HUMAN | O60682 | 2 | 3 | 1 | | 2 | |
| 67 | 60S ribosomal protein L19 - Homo sapiens (Human) | RL19_HUMAN | P84098 | | 1 | 2 | 2 | 1 | 3 |
| 68 | Stress-70 protein, mitochondrial precursor (75 kDa glucose-regulated protein) (GRP 75) (Peptide-binding | GRP75_HUMAN | P38646 | 6 | 6 | 2 | 7 | 3 | 5 |
| | protein 74) (PBP74) (Mortalin) (MOT) - Homo sapiens (Human) | | | | | | | | |
| 69 | Ubiquinol-cytochrome-c reductase complex core protein I, mitochondrial precursor (EC 1.10.2.2) - Homo | UQCR1_HUMAN | P31930 | | 1 | 3 | 2 | 2 | |
| | sapiens (Human) | | | | | | | | |
| 70 | 60S ribosomal protein L22 (Epstein-Barr virus small RNA-associated protein) (EBER-associated protein) (EAP) (Heparin- | RL22_HUMAN | P35268 | | 1 | | | 1 | 1 |
| | binding protein HBp15) - Homo sapiens (Human) | | | | | | | | |
| 71 | Ras-related protein Rab-7 - Homo sapiens (Human) | RAB7_HUMAN | P51149 | 3 | 1 | 3 | 1 | 3 | 4 |
| 72 | KIAA0466 protein (Fragment) - Homo sapiens (Human) | O75054_HUMAN | O75054 | 1 | | 1 | 1 | 1 | |
| 73 | Macrophage capping protein (Actin-regulatory protein CAP-G) - Homo sapiens (Human) | CAPG_HUMAN | P40121 | | 2 | | 1 | 1 | 3 |
| 74 | Small nuclear ribonucleoprotein Sm D1 (snRNP core protein D1) (Sm-D1) (Sm-D autoantigen) - Homo sapiens | SMD1_HUMAN | P62314 | 2 | 1 | 3 | 2 | 1 | 1 |
| | (Human) | | | | | | | | |
| 75 | T-complex protein 1 subunit zeta (TCP-1-zeta) (CCT-zeta) (CCT-zeta-1) (Tcp20) (HTR3) (Acute morphine | TCPZ_HUMAN | P40227 | 3 | 2 | 2 | 1 | 3 | 4 |
| | dependence-related protein 2) - Homo sapiens (Human) | | | | | | | | |
| 76 | Talin-1 - Homo sapiens (Human) | TLN1_HUMAN | Q9Y490 | 7 | 15 | 9 | 13 | 11 | 14 |
| 77 | Basement membrane-specific heparan sulfate proteoglycan core protein precursor (HSPG) (Perlecan) (PLC) - | PGBM_HUMAN | P98160 | 5 | 11 | 6 | 5 | 7 | 4 |
| | Homo sapiens (Human) | | | | | | | | |
| 78 | Cytoglobin (Histoglobin) (HGb) (Stellate cell activation-associated protein) - Homo sapiens (Human) | CYGB_HUMAN | Q8WWM9 | | | 1 | | 1 | 1 |
| 79 | 60S ribosomal protein L13 (Breast basic conserved protein 1) - Homo sapiens (Human) | RL13_HUMAN | P26373 | 3 | 1 | 2 | 3 | 3 | 4 |
| 80 | Plasminogen activator inhibitor 1 RNA-binding protein (PAI1 RNA-binding protein 1) (PAI-RBP1) | PAIRB_HUMAN | Q8NC51 | 8 | 6 | 5 | 5 | 4 | 5 |
| | (SERPINE1 mRNA-binding protein 1) - Homo sapiens (Human) | | | | | | | | |
| 81 | Protein bassoon (Zinc finger protein 231) - Homo sapiens (Human) | BSN_HUMAN | Q9UPA5 | 5 | 3 | 4 | 2 | 3 | 2 |
| 82 | Aspartate aminotransferase, mitochondrial precursor (EC 2.6.1.1) (Transaminase A) (Glutamate oxaloacetate | AATM_HUMAN | P00505 | 4 | 4 | 3 | 4 | | 2 |
| | transaminase 2) - Homo sapiens (Human) | | | | | | | | |
| 83 | Complement component 1 Q subcomponent-binding protein, mitochondrial precursor (Glycoprotein gC1qBP) | C1QBP_HUMAN | Q07021 | 2 | 1 | 1 | 2 | 3 | 1 |
| | (C1qBP) (GC1q-R protein) (Hyaluronan-binding protein 1) (Mitochondrial matrix protein p32) (p33) - Homo | | | | | | | | |
| 84 | GTP-binding nuclear protein Ran (GTPase Ran) (Ras-like protein TC4) (Androgen receptor-associated protein | RAN_HUMAN | P62826 | 1 | 3 | 2 | 3 | 1 | |
| | 24) - Homo sapiens (Human) | | | | | | | | |
| 85 | Anaphase-promoting complex subunit 10 (APC10) (Cyclosome subunit 10) - Homo sapiens (Human) | APC10_HUMAN | Q9UM13 | | 1 | | | | |
| 86 | Transgelin (Smooth muscle protein 22-alpha) (SM22-alpha) (WS3-10) (22 kDa actin-binding protein) - Homo | TAGL_HUMAN | Q01995 | | 5 | | 5 | 5 | 4 |
| | sapiens (Human) | | | | | | | | |
| 87 | 40S ribosomal protein S6 (Phosphoprotein NP33) - Homo sapiens (Human) | RS6_HUMAN | P62753 | 1 | 1 | 2 | 1 | 3 | 3 |
| 88 | NADH-cytochrome b5 reductase (EC 1.6.2.2) (B5R) (Diaphorase-1) (Cytochrome b5 reductase 3) [Contains: | NCB5R_HUMAN | P00387 | 3 | 2 | 2 | 1 | 2 | 2 |
| | NADH-cytochrome b5 reductase membrane-bound form; NADH-cytochrome b5 reductase soluble form] - | | | | | | | | |
| 89 | CDNA: FLJ21665 fis, clone COL08913 (Tubulin, alpha-like 3) - Homo sapiens (Human) | Q9H6Z0_HUMA | Q9H6Z0 | | 1 | | 1 | | 1 |
| 90 | Serpin H1 precursor (Collagen-binding protein) (Colligin) (47 kDa heat shock protein) (Rheumatoid arthritis- | SERPH_HUMAN | P50454 | 1 | | | 2 | | |
| | related antigen RA-A47) (Arsenic-transactivated protein 3) (AsTP3) (Proliferation-inducing gene 14 protein) - | | | | | | | | |
| 91 | Myosin light polypeptide 6 (Smooth muscle and nonmuscle myosin light chain alkali 6) (Myosin light chain | MYL6_HUMAN | P60660 | 3 | 4 | | 1 | 1 | 2 |
| | alkali 3) (Myosin light chain 3) (MLC-3) (LC17) - Homo sapiens (Human) | | | | | | | | |
| 92 | Glycogen phosphorylase, brain form (EC 2.4.1.1) - Homo sapiens (Human) | PYGB_HUMAN | P11216 | 1 | 3 | 2 | 3 | 2 | 2 |
| 93 | Single-stranded DNA-binding protein, mitochondrial precursor (Mt-SSB) (MtSSB) (PWP1-interacting protein | SSB_HUMAN | Q04837 | | 1 | | 1 | 1 | 2 |
| | 17) - Homo sapiens (Human) | | | | | | | | |
| 94 | Adipocyte plasma membrane-associated protein (BSCv protein) - Homo sapiens (Human) | APMAP_HUMAN | Q9HDC9 | | | 2 | 2 | 2 | 1 |
| 95 | Voltage-dependent anion-selective channel protein 1 (VDAC-1) (hVDAC1) (Outer mitochondrial membrane | VDAC1_HUMAN | P21796 | | | 1 | | | 2 |
| | protein porin 1) (Plasmalemmal porin) (Porin 31HL) (Porin 31HM) - Homo sapiens (Human) | | | | | | | | |
| 96 | Coatomer subunit beta (Beta-coat protein) (Beta-COP) - Homo sapiens (Human) | COPB_HUMAN | P53618 | 2 | | 3 | 1 | 1 | 1 |
| 97 | Hypothetical protein DKFZp686C0331 - Homo sapiens (Human) | Q68DY1_HUMA | Q68DY1 | | 1 | | | | |
| 98 | Annexin A3 (Annexin III) (Lipocortin III) (Placental anticoagulant protein III) (PAP-III) (35-alpha calcimedin) | ANXA3_HUMAN | P12429 | 2 | 3 | 2 | 1 | 4 | 1 |
| | (Inositol 1,2-cyclic phosphate 2-phosphohydrolase) - Homo sapiens (Human) | | | | | | | | |
| 99 | Splicing factor, arginine/serine-rich 1 (pre-mRNA-splicing factor SF2, P33 subunit) (Alternative-splicing factor | SFRS1_HUMAN | Q07955 | 6 | 6 | 5 | 5 | 5 | 4 |
| | 1) (ASF-1) - Homo sapiens (Human) | | | | | | | | |
| 100 | Plastin-2 (L-plastin) (Lymphocyte cytosolic protein 1) (LCP-1) (LC64P) - Homo sapiens (Human) | PLSL_HUMAN | P13796 | 1 | 4 | 5 | 4 | 1 | 5 |
| 101 | Growth-inhibiting protein 12 - Homo sapiens (Human) | Q5DSM0_HUMA | Q5DSM0 | 5 | 5 | 5 | 16 | 5 | |
| 102 | Protein S100-A8 (S100 calcium-binding protein A8) (Calgranulin-A) (Migration inhibitory factor-related | S10A8_HUMAN | P05109 | | 4 | | 1 | 1 | |
| | protein 8) (MRP-8) (Cystic fibrosis antigen) (CFAG) (P8) (Leukocyte L1 complex light chain) (Calprotectin | | | | | | | | |
| 103 | Forkhead box protein D2 (Forkhead-related protein FKHL17) (Forkhead-related transcription factor 9) (FREAC- | FOXD2_HUMAN | O60548 | 1 | | | 2 | 3 | |
| | 9) - Homo sapiens (Human) | | | | | | | | |
| 104 | OTU domain-containing protein 6B - Homo sapiens (Human) | OTU6B_HUMAN | Q8N6M0 | 1 | 1 | 1 | 1 | | |
| 105 | Fatty acid-binding protein, epidermal (E-FABP) (Psoriasis-associated fatty acid-binding protein homolog) (PA- | FABPE_HUMAN | Q01469 | | | | | 1 | |
| | FABP) - Homo sapiens (Human) | | | | | | | | |
| 106 | Ventral anterior homeobox 1 - Homo sapiens (Human) | VAX1_HUMAN | Q5SQQ9 | | | | | | |
| 107 | DPYSL3 protein - Homo sapiens (Human) | Q6DEN2_HUMA | Q6DEN2 | 1 | 2 | 1 | 1 | 2 | 2 |
| 108 | Peroxisomal multifunctional enzyme type 2 (MFE-2) (D-bifunctional protein) (DBP) (17-beta-hydroxysteroid | DHB4_HUMAN | P51659 | 8 | | 1 | 5 | 3 | 1 |
| | dehydrogenase 4) (17-beta-HSD 4) (D-3-hydroxyacyl-CoA dehydratase) (EC 4.2.1.107) (3-alpha,7-alpha,12- | | | | | | | | |
| 109 | Rootletin (Ciliary rootlet coiled-coil protein) - Homo sapiens (Human) | CROCC_HUMA | Q5TZA2 | | | | | 2 | |
| 110 | AFG3-like protein 2 (EC 3.4.24.- ) (Paraplegin-like protein) - Homo sapiens (Human) | AFG32_HUMAN | Q9Y4W6 | 4 | 2 | 1 | 4 | 3 | 2 |
| 111 | Collagen alpha-3(IV) chain precursor (Goodpasture antigen) - Homo sapiens (Human) | CO4A3_HUMAN | Q01955 | 1 | 3 | 1 | 4 | 1 | |
| 112 | Cell death regulator Aven - Homo sapiens (Human) | AVEN_HUMAN | Q9NQS1 | | | 1 | 2 | 2 | |
| 113 | 60S acidic ribosomal protein P0 (L10E) - Homo sapiens (Human) | RLA0_HUMAN | P05388 | | | | | | |
| 114 | Collagen alpha-1(VII) chain precursor (Long-chain collagen) (LC collagen) - Homo sapiens (Human) | CO7A1_HUMAN | Q02388 | 4 | 4 | 4 | 2 | | 1 |
| 115 | Histone H1.0 (H1(0)) (Histone H1′) - Homo sapiens (Human) | H10_HUMAN | P07305 | 4 | 2 | 5 | 2 | 2 | 5 |
| 116 | ERGIC-53 protein precursor (ER-Golgi intermediate compartment 53 kDa protein) (Lectin, mannose-binding 1) | LMAN1_HUMAN | P49257 | | | 1 | 2 | 1 | 2 |
| | (Gp58) (Intracellular mannose-specific lectin MR60) - Homo sapiens (Human) | | | | | | | | |
| 117 | Lupus La protein (Sjoegren syndrome type B antigen) (SS-B) (La ribonucleoprotein) (La autoantigen) - Homo | LA_HUMAN | P05455 | 1 | | 2 | 1 | 2 | 1 |
| | sapiens (Human) | | | | | | | | |
| 118 | LIM and SH3 domain protein 1 (LASP-1) (MLN 50) - Homo sapiens (Human) | LASP1_HUMAN | Q14847 | 2 | 4 | 1 | | 2 | |
| 119 | Bifunctional purine biosynthesis protein PURH [Includes: Phosphoribosylaminoimidazolecarboxamide | PUR9_HUMAN | P31939 | 1 | 2 | 4 | 1 | 4 | 1 |
| | formyltransferase (EC 2.1.2.3) (AICAR transformylase); IMP cyclohydrolase (EC 3.5.4.10) (Inosinicase) (IMP | | | | | | | | |
| 120 | Non-POU domain-containing octamer-binding protein (NonO protein) (54 kDa nuclear RNA- and DNA-binding | NONO_HUMAN | Q15233 | 5 | 4 | 4 | 5 | 5 | 7 |
| | protein) (p54(nrb)) (p54nrb) (55 kDa nuclear protein) (NMT55) (DNA-binding p52/p100 complex, 52 kDa | | | | | | | | |
| 121 | Alpha-actinin-4 (Non-muscle alpha-actinin 4) (F-actin cross-linking protein) - Homo sapiens (Human) | ACTN4_HUMAN | O43707 | 6 | 6 | 7 | 6 | 7 | 10 |
| 122 | Very-long-chain specific acyl-CoA dehydrogenase, mitochondrial precursor (EC 1.3.99.- ) (VLCAD) - Homo | ACADV_HUMAN | P49748 | | 5 | 5 | 3 | 3 | 3 |
| | sapiens (Human) | | | | | | | | |
| 123 | Xylosyltransferase 1 (EC 2.4.2.26) (Xylosyltransferase I) (XylT-I) (XT-I) (Peptide O-xylosyltransferase 1) - | XYLT1 _HUMAN | Q86Y38 | 1 | | 3 | 1 | 2 | 2 |
| | Homo sapiens (Human) | | | | | | | | |
| 124 | Proteasome subunit alpha type 1 (EC 3.4.25.1) (Proteasome component C2) (Macropain subunit C2) | PSA1_HUMAN | P25786 | 1 | 1 | 1 | 3 | | 1 |
| | (Multicatalytic endopeptidase complex subunit C2) (Proteasome nu chain) (30 kDa prosomal protein) (PROS- | | | | | | | | |
| 125 | Baculoviral IAP repeat-containing protein 6 (Ubiquitin-conjugating BIR-domain enzyme apollon) - Homo | BIRC6_HUMAN | Q9NR09 | 1 | 3 | | | 2 | 1 |
| | sapiens (Human) | | | | | | | | |
| 126 | Mannosyl-oligosaccharide glucosidase (EC 3.2.1.106) (Processing A-glucosidase I) - Homo sapiens (Human) | GCS1_HUMAN | Q13724 | 1 | | 4 | 1 | | 1 |
| 127 | (Poly [ADP-ribose] polymerase 1 (EC 2.4.2.30) (PARP-I) (ADPRT) (NAD(+) ADP-ribosyltransferase 1) | PARP1_HUMAN | P09874 | 1 | 2 | 4 | 1 | 2 | 1 |
| | Poly[ADP-ribose] synthetase 1) - Homo sapiens (Human) | | | | | | | | |
| 128 | Ezrin-radixin-moesin-binding phosphoprotein 50 (EBP50) (Na(+)/H(+) exchange regulatory cofactor NHE-RF) | NHERF_HUMAN | O14745 | 1 | 1 | 2 | 1 | 3 | 2 |
| | (NHERF-1) (Regulatory cofactor of Na(+)/H(+) exchanger) (Sodium-hydrogen exchanger regulatory factor 1) | | | | | | | | |
| 129 | CDNA FLJ46519 fis, clone THYMU3033649, highly similar to T-cell surface glycoprotein CD3 zeta chain - | Q6KAV0_HUMAN | Q6KAV0 | 1 | | | 2 | 1 | 1 |
| | Homo sapiens (Human) | | | | | | | | |
| 130 | Ornithine decarboxylase antizyme 2 (ODC-Az 2) (AZ2) - Homo sapiens (Human) | OAZ2_HUMAN | O95190 | 1 | | 1 | | 1 | 1 |
| 131 | Thioredoxin domain-containing protein 4 precursor (Endoplasmic reticulum resident protein ERp44) - Homo | TXND4_HUMAN | Q9BS26 | | 1 | 3 | 4 | | |
| | sapiens (Human) | | | | | | | | |
| 132 | N-acetylated-alpha-linked acidic dipeptidase 2 (EC 3.4.17.21) (N-acetylated-alpha-linked acidic dipeptidase II) | NALD2_HUMAN | Q9Y3Q0 | | | 1 | | | 1 |
| | (NAALADase II) - Homo sapiens (Human) | | | | | | | | |
| 122 | CDNA FLJ23660 fis, clone HEP00931 (Phospholipase C, delta 3) - Homo sapiens (Human) | Q8TEC1_HUMA | Q8TEC1 | | | | 1 | | |
| 134 | Novel protein similar to histone 2, H3c (HIST2H3C) - Homo sapiens (Human) | Q5TEC6_HUMA | Q5TEC6 | | 1 | | | | |
| 135 | Cyclin M3 (Hypothetical protein CNNM3) - Homo sapiens (Human) | Q8NE01_HUMA | Q8NE01 | | | | 1 | | |
| 136 | 4-trimethylaminobutyraldehyde dehydrogenase (EC 1.2.1.47) (TMABADH) (Aldehyde dehydrogenase 9A1) | AL9A1_HUMAN | P49189 | 2 | 4 | 1 | 3 | 2 | |
| | (EC 1.2.1.3) (Aldehyde dehydrogenase E3 isozyme) (Gamma-aminobutyraldehyde dehydrogenase) (EC | | | | | | | | |
| 137 | Netrin receptor UNC5D precursor (Unc-5 homolog D) (Unc-5 homolog 4) - Homo sapiens (Human) | UNC5D_HUMAN | Q6UXZ4 | 2 | 1 | | 1 | | |
| 138 | Protein S100-A11 (S100 calcium-binding protein A11) (Protein S100C) (Calgizzarin) (MLN 70) - Homo | S10AB_HUMAN | P31949 | | | 2 | 2 | 1 | |
| | sapiens (Human) | | | | | | | | |
| 139 | Pyruvate carboxylase, mitochondrial precursor (EC 6.4.1.1) (Pyruvic carboxylase) (PCB) - Homo sapiens | PYC_HUMAN | P11498 | 1 | | | | 1 | |
| | (Human) | | | | | | | | |
| 140 | Zinc finger and SCAN domain-containing protein 4 (Zinc finger protein 494) - Homo sapiens | ZSCA4_HUMAN | Q8NAM6 | | | | | | |

| | Column | | | | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|---|---|---|---|
| | 10 | 11 | 12 | 13 | 14 | 15 | 16 | 17 | 18 | 19 | 20 | 21 | 22 | 23 | 24 | 25 | 26 | 27 | 28 | 29 | 30 | 31 | 32 | 33 | 34 | 35 | 36 | 37 | 38 | 39 | 40 | 41 | 42 | 43 | 44 | 45 |
| | Sample# | | | | | | | | | | | | | | | | | | | | | | | | | | | | | | | | | | | |
| Row | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 | 31 | 32 | 33 | 34 | 35 | 36 | 37 | 38 | 39 | 40 | 41 | 42 |
| 1 | 7 | 5 | 5 | 2 | 2 | 5 | 2 | 2 | 2 | 4 | 3 | 3 | 6 | 6 | 1 | 4 | 2 | 2 | 1 | 3 | 2 | 6 | 2 | 1 | 4 | 2 | 2 | | 2 | 3 | 1 | 0 | 0 | 1 | 3 | 0 |
| 2 | 6 | 8 | 7 | 9 | 5 | 7 | 3 | 2 | 3 | 4 | 3 | 3 | 6 | 10 | 1 | 9 | 8 | 1 | 2 | 8 | 2 | 7 | 7 | 6 | 10 | 8 | 9 | 2 | 2 | 3 | 3 | 2 | 3 | 3 | 4 | 0 |
| 3 | 8 | 7 | 10 | 5 | 5 | 6 | 2 | 4 | 3 | 2 | 1 | 2 | 4 | 2 | 2 | 7 | 8 | 1 | 1 | 8 | 2 | 5 | 5 | 3 | 8 | 7 | 2 | 2 | 1 | 2 | 2 | 2 | 2 | 2 | 3 | 2 |
| 4 | 7 | 4 | 7 | 2 | 5 | 5 | 2 | 1 | 1 | 3 | 3 | 3 | 7 | 11 | 1 | 8 | 9 | 1 | 0 | 12 | 3 | 6 | 3 | 4 | 8 | 5 | 2 | 1 | 1 | 0 | 1 | 2 | 3 | 2 | 1 | 0 |
| 5 | 8 | 2 | 2 | 6 | 1 | 5 | 1 | 2 | 2 | 4 | 3 | 5 | 2 | 3 | 2 | 3 | 3 | 2 | 1 | 7 | 1 | 3 | 3 | 2 | 2 | 3 | 0 | 2 | 2 | 1 | 0 | 1 | 2 | 2 | 2 | 1 |
| 6 | 14 | 12 | 10 | 11 | 10 | 9 | 3 | | 5 | 4 | 2 | 2 | 5 | 2 | 2 | 2 | 3 | 1 | 0 | 2 | 1 | 3 | 1 | 2 | 1 | 1 | 1 | 1 | 2 | 1 | 0 | 0 | 2 | 1 | 2 | 0 |
| 7 | 7 | 4 | 5 | 7 | 5 | 5 | 4 | 3 | 3 | 6 | 5 | 3 | 6 | 5 | | 4 | 2 | 2 | 3 | 5 | 3 | 4 | 3 | 0 | 3 | 2 | 3 | 4 | 3 | 2 | 0 | 1 | 3 | 1 | 2 | 0 |
| 8 | 1 | 4 | 5 | | 4 | 6 | 2 | 1 | 1 | 1 | 3 | 2 | 4 | 2 | 1 | 5 | 3 | 2 | 0 | 3 | 4 | 4 | 2 | 3 | 3 | 3 | 0 | 2 | 1 | 0 | 0 | 1 | 1 | 3 | 2 | 1 |
| 9 | 4 | 2 | 5 | 2 | 4 | 3 | | | 1 | 3 | 2 | 1 | 5 | 4 | 1 | 11 | 7 | 2 | 1 | 4 | 3 | 4 | 5 | 5 | 10 | 3 | 2 | 2 | | 2 | 2 | 1 | 4 | 3 | 3 | 1 |
| 10 | 5 | 9 | 7 | 6 | 4 | 5 | 2 | 1 | | 2 | 3 | 3 | 6 | 6 | 1 | 6 | 7 | 4 | 2 | 5 | 0 | 6 | 5 | 1 | 1 | 6 | 0 | 1 | | 2 | 0 | 0 | 3 | 3 | 0 | 0 |
| 11 | 2 | 4 | 3 | 3 | 6 | 4 | 1 | 3 | 1 | 4 | 2 | 2 | 6 | 11 | 1 | 4 | 1 | 0 | 0 | 9 | 0 | 5 | 1 | 11 | 3 | 4 | 0 | | | 1 | 0 | 1 | 1 | 1 | 2 | 0 |
| 12 | 22 | 12 | 11 | 7 | 14 | 14 | 3 | 9 | 4 | 10 | 8 | 9 | 6 | 9 | | 4 | 16 | 1 | 0 | 4 | 1 | 3 | 1 | 2 | 0 | 4 | 0 | 4 | 1 | 0 | 0 | 2 | 0 | 2 | 3 | 0 |
| 13 | 5 | 3 | 6 | 2 | 2 | 2 | 1 | 3 | 1 | 1 | 3 | 2 | 0 | 3 | | 2 | 2 | 1 | 1 | 2 | 1 | 4 | 2 | 0 | 2 | 1 | 0 | 1 | 2 | 2 | 0 | 1 | 0 | 0 | 1 | 0 |
| 14 | 9 | 9 | 2 | 5 | 6 | 6 | 2 | 1 | | 3 | 2 | 1 | 9 | 4 | | 5 | 5 | 0 | 1 | 6 | 1 | 2 | 3 | 1 | 2 | 2 | 0 | 1 | 1 | 2 | 0 | 3 | 2 | 2 | 0 | 0 |
| 15 | 2 | 2 | 1 | | 1 | 1 | 1 | 1 | 1 | 2 | 1 | 2 | 3 | 3 | 1 | 2 | 0 | 0 | 0 | 4 | 1 | 2 | 0 | 1 | 2 | 1 | 0 | 1 | 1 | 0 | 1 | 0 | 1 | 0 | 0 | 0 |
| 16 | 43 | 6 | 11 | 1 | 6 | 10 | 1 | 3 | 2 | 1 | 9 | 1 | 17 | 7 | | 3 | 3 | 7 | 2 | 2 | 0 | 1 | 1 | 3 | 0 | 0 | 0 | 1 | 4 | 0 | 0 | 0 | 5 | 3 | 2 | 0 |
| 17 | 1 | 2 | | | | 1 | | 1 | | 1 | | 2 | 1 | 1 | | 1 | 1 | 1 | 2 | 1 | 2 | 1 | 1 | 1 | 1 | 1 | 0 | 1 | | 1 | 0 | 1 | 1 | 1 | 0 | 0 |
| 18 | | 2 | 1 | 1 | 1 | 1 | | | | 1 | | | 1 | 2 | | 1 | 1 | 1 | 1 | 1 | 1 | 1 | 2 | 1 | 3 | 1 | 1 | 1 | | 0 | 0 | 0 | 0 | 0 | 0 | 0 |
| 19 | 2 | 1 | 1 | 5 | 2 | 1 | | 1 | | 1 | 1 | 2 | 2 | 5 | | 5 | 2 | 0 | 1 | 5 | 0 | 2 | 3 | 4 | 5 | 0 | 4 | 1 | 1 | 0 | 1 | 0 | 0 | 0 | 0 | 0 |
| 20 | 5 | 5 | 4 | 2 | 4 | 4 | 1 | 1 | 2 | 2 | 2 | 3 | 3 | 4 | | 2 | 1 | 0 | 0 | 6 | 0 | 2 | 1 | 0 | 2 | 1 | 0 | 1 | 1 | 0 | 0 | 1 | 2 | 0 | 1 | 0 |
| 21 | 3 | 3 | 2 | 3 | 2 | 3 | 1 | 1 | 1 | 1 | 1 | 1 | 4 | 1 | 1 | 4 | 2 | 0 | 0 | 4 | 0 | 2 | 1 | 0 | 0 | 1 | 0 | 1 | 1 | 0 | 0 | 1 | 1 | 0 | 1 | 0 |
| 22 | 16 | 16 | 12 | 7 | 6 | 11 | 1 | 2 | 2 | 6 | 5 | 5 | 2 | 4 | | 3 | 0 | 1 | 0 | 0 | 0 | 1 | 3 | 1 | 2 | 3 | 0 | 1 | | 2 | 0 | 2 | 2 | 1 | 0 | 0 |
| 23 | 5 | 6 | 8 | 4 | 1 | 4 | 1 | 2 | | 2 | 3 | 3 | 2 | 2 | 1 | 5 | 5 | 0 | 0 | 4 | 0 | 2 | 0 | 1 | 1 | 1 | 0 | 3 | 1 | 1 | 0 | 0 | 2 | 1 | 1 | 0 |
| 24 | 3 | 3 | 4 | 4 | 3 | 2 | 2 | 1 | 2 | 1 | 3 | 2 | 5 | 9 | | 0 | 1 | 0 | 0 | 0 | 6 | 4 | 9 | 5 | 0 | 3 | 3 | 1 | 1 | 5 | 0 | 2 | 1 | 1 | 2 | 0 |
| 25 | 9 | 9 | 10 | 12 | 8 | 8 | 1 | | | 4 | 3 | 4 | 8 | 6 | | 10 | 7 | 1 | 0 | 6 | 0 | 8 | 3 | 1 | 1 | 2 | 0 | 1 | | 3 | 0 | 2 | 3 | 5 | 3 | 0 |
| 26 | | 1 | 1 | 1 | 1 | 1 | 2 | 2 | 1 | 2 | 2 | 2 | 1 | 1 | 1 | 3 | 0 | 0 | 0 | 1 | 0 | 0 | 1 | 0 | 2 | 1 | 0 | | | 0 | 0 | 0 | 0 | 0 | 0 | 0 |
| 27 | 21 | 6 | 6 | | 1 | 4 | 2 | 3 | 2 | 5 | 1 | 1 | 1 | 2 | | 2 | 5 | 0 | 0 | 4 | 0 | 0 | 6 | 1 | 0 | 1 | 0 | | | 0 | 0 | 0 | 0 | 0 | 0 | 0 |
| 28 | 2 | 3 | 1 | 1 | | 3 | 1 | | | | | 2 | 1 | 1 | | 1 | 1 | 0 | 1 | 1 | 1 | 1 | 1 | 1 | 1 | 0 | 1 | | | 0 | 1 | 0 | 0 | 0 | 0 | 0 |
| 29 | 1 | 1 | 1 | 1 | 1 | 2 | | 1 | | 1 | 1 | 1 | 0 | 1 | 1 | 1 | 1 | 1 | 1 | 0 | 0 | 1 | 1 | 1 | 1 | 0 | 0 | | | 0 | 0 | 0 | 0 | 0 | 0 | 0 |
| 30 | 2 | 2 | 1 | 3 | 1 | 1 | 1 | 1 | 1 | 2 | 1 | 1 | 1 | 2 | 1 | 0 | 0 | 0 | 0 | 1 | 0 | 1 | 0 | 1 | 1 | 1 | 0 | | | 0 | 0 | 1 | 1 | 0 | 0 | 0 |
| 31 | 1 | 1 | 3 | 2 | 1 | 1 | | | | 4 | 1 | 2 | 1 | 3 | | 5 | 2 | 1 | 1 | 0 | 1 | 2 | 3 | 0 | 2 | 2 | 0 | | | 0 | 0 | 0 | 1 | 1 | 2 | 0 |
| 32 | 2 | 3 | 1 | 1 | | | 2 | 1 | | 1 | 1 | 1 | 1 | 1 | | 4 | 1 | 0 | 0 | 3 | 0 | 2 | 0 | 1 | 2 | 1 | 0 | | | 1 | 0 | 0 | 0 | 2 | 0 | 0 |
| 33 | 5 | 4 | 5 | 3 | 3 | 4 | | 2 | 1 | 3 | 2 | 1 | 2 | 3 | | 5 | 3 | 0 | 0 | 5 | 0 | 2 | 1 | 0 | 5 | 1 | 0 | | 1 | 0 | 1 | 1 | 0 | 0 | 1 | 0 |
| 34 | 4 | 7 | 10 | 5 | 4 | 4 | 1 | | 1 | 5 | 3 | 3 | 5 | 4 | 1 | 4 | 3 | | | 3 | 1 | | | 2 | 3 | | | 1 | 2 | 1 | | | 1 | 1 | | |
| 35 | 3 | 2 | 2 | 1 | 2 | 1 | 1 | | 1 | 2 | 2 | | 1 | 2 | 1 | 1 | 0 | 2 | 2 | 0 | 0 | 2 | 1 | 1 | 2 | 0 | 0 | 1 | | 2 | 0 | 0 | 1 | 0 | 0 | 0 |
| 36 | 2 | 2 | | | 3 | 1 | 1 | | 1 | | | 1 | 0 | 2 | | 2 | 0 | 1 | 1 | 3 | 1 | 1 | 1 | 1 | 0 | 2 | 1 | | | 0 | 0 | 1 | 1 | 1 | 0 | 0 |
| 37 | 6 | 4 | 3 | 3 | 3 | 7 | 1 | | 2 | 1 | 1 | 2 | 3 | 3 | | 1 | 0 | 0 | 0 | 3 | 0 | 4 | 2 | 1 | 2 | 1 | 0 | 1 | 1 | 1 | 0 | 1 | 0 | 1 | 1 | 0 |
| 38 | 3 | 2 | 3 | 3 | | | | 1 | 2 | 3 | 1 | 1 | 1 | 2 | | 3 | 2 | 0 | 0 | 4 | 0 | 1 | 0 | 2 | 2 | 2 | 0 | | 1 | 1 | 1 | 1 | 1 | 0 | 0 | 0 |
| 39 | 4 | | 1 | 1 | | 2 | 2 | 1 | 1 | 1 | 2 | | 2 | 2 | | 2 | 2 | 1 | 0 | 2 | 0 | 0 | 1 | 0 | 0 | 1 | 1 | 2 | 1 | 0 | 0 | 1 | 0 | 1 | 1 | 0 |
| 40 | 4 | 4 | 2 | | 3 | 6 | | | | 1 | 1 | 1 | 6 | 7 | | 0 | 0 | 2 | 2 | 1 | 3 | 1 | 0 | 1 | 4 | 3 | 0 | | | 0 | 0 | 0 | 0 | 0 | 0 | 0 |
| 41 | 14 | 6 | 1 | | 8 | 6 | | 5 | 1 | 2 | 1 | 2 | 8 | 4 | | 1 | 8 | 1 | 0 | 1 | 0 | 0 | 1 | 0 | 0 | 2 | 0 | | 1 | 0 | 0 | 0 | 1 | 0 | 0 | 0 |
| 42 | 1 | 1 | | | | 1 | | | 1 | 1 | | | 3 | 0 | | 1 | 0 | 2 | 2 | 0 | 1 | 1 | 1 | 1 | 2 | 1 | 1 | | | 0 | 0 | 0 | 1 | 0 | 1 | 0 |
| 43 | 4 | 4 | | 2 | 1 | 3 | 1 | | 2 | 2 | | 1 | 2 | 1 | | 0 | 0 | 0 | 1 | 1 | 1 | 1 | 1 | 0 | 1 | 0 | 1 | 3 | | 0 | 0 | 0 | 0 | 0 | 0 | 0 |
| 44 | 4 | 6 | 6 | 4 | 3 | 2 | | 1 | | 2 | 1 | 1 | 0 | 2 | | 7 | 7 | 0 | 0 | 3 | 0 | 2 | 1 | 2 | 1 | 4 | 0 | 1 | | 1 | 0 | 1 | 2 | 0 | 1 | 0 |
| 45 | 2 | 2 | 2 | 2 | 2 | 2 | 1 | 1 | 1 | 1 | 1 | 1 | 1 | 1 | 1 | 1 | | | | 1 | | | | | 1 | 1 | | 1 | 1 | | | | 1 | | | |
| 46 | 4 | 2 | 2 | 3 | 3 | | 1 | 2 | 1 | 1 | | 3 | 2 | 1 | | 2 | 1 | 1 | 0 | 1 | 0 | 2 | 1 | 0 | 0 | 0 | 0 | 1 | 2 | 0 | 0 | 0 | 1 | 1 | 0 | 0 |
| 47 | 1 | | 5 | | | 1 | 1 | | 2 | 2 | 1 | | 5 | 1 | | 0 | 1 | 1 | 1 | 0 | 0 | 0 | 1 | 1 | 0 | 1 | 0 | | | 0 | 0 | 0 | 0 | 1 | 0 | 0 |
| 48 | 1 | | 2 | | 1 | | 1 | 1 | | 1 | 1 | 1 | 2 | 2 | | 3 | 1 | 0 | 0 | 1 | 0 | 0 | 1 | 0 | 1 | 0 | 0 | | 1 | 0 | 0 | 0 | 0 | 0 | 0 | 0 |
| 49 | 3 | 3 | 5 | 2 | 3 | 3 | 1 | | 1 | | 2 | 2 | 3 | 3 | 1 | 3 | 0 | 0 | 0 | 0 | 0 | 1 | 2 | 1 | 0 | 1 | 0 | 1 | | 0 | 0 | 0 | 1 | 0 | 0 | 0 |
| 50 | 5 | 5 | 3 | 1 | 2 | 2 | 1 | 1 | | 4 | 1 | 2 | 1 | 4 | | 3 | 4 | 0 | 0 | 1 | 0 | 0 | 0 | 1 | 0 | 1 | 0 | | | 0 | 0 | 2 | 1 | 0 | 0 | 0 |
| 51 | 8 | 10 | 5 | 7 | 7 | 4 | 2 | 3 | 4 | 5 | 4 | 4 | 2 | 0 | 1 | 2 | 3 | 0 | 0 | 2 | 0 | 1 | 0 | 0 | 0 | 0 | 0 | 1 | 3 | 0 | 0 | 0 | 1 | 0 | 0 | 0 |
| 52 | | | 6 | 4 | | 1 | 1 | 5 | 6 | 3 | 4 | 3 | 0 | 0 | 1 | 1 | 0 | 3 | 0 | 0 | 0 | 0 | 3 | 0 | 0 | 1 | 2 | | 2 | 0 | 0 | 1 | 1 | 1 | 0 | 0 |
| 53 | 7 | 8 | 6 | 3 | 2 | 2 | | | 1 | 4 | | 1 | 2 | 2 | | 1 | 1 | 0 | 1 | 2 | 0 | 1 | 1 | 0 | 0 | 2 | 0 | | | 1 | 0 | 1 | 0 | 0 | 1 | 0 |
| 54 | 3 | 3 | 2 | 2 | 3 | 4 | 1 | 1 | 1 | 2 | | 1 | 3 | 2 | | 2 | 3 | 0 | 0 | 1 | 0 | 1 | 1 | 0 | 0 | 0 | 0 | | 1 | 0 | 0 | 0 | 0 | 0 | 0 | 0 |
| 55 | 5 | 4 | 5 | 1 | | | 2 | 1 | 1 | 2 | 4 | 1 | 1 | 0 | | 1 | 0 | 0 | 0 | 0 | 1 | 3 | 1 | 0 | 0 | 1 | 0 | 2 | | 1 | 0 | 0 | 0 | 0 | 0 | 0 |
| 65 | 4 | 3 | 3 | | 2 | | | 1 | 1 | 2 | | 2 | 2 | 1 | | 2 | 1 | 0 | 0 | 2 | 1 | 3 | 0 | 0 | 0 | 1 | 0 | 2 | 1 | 1 | 0 | 0 | 0 | 0 | 0 | 0 |
| 57 | 4 | | | | | | 1 | | | | | | 1 | 0 | | 1 | 0 | 1 | 1 | 2 | 1 | 1 | 1 | 1 | 1 | 1 | 0 | 1 | | 0 | 1 | 0 | 0 | 0 | 0 | 0 |
| 58 | 8 | 4 | 2 | 10 | 1 | 13 | 1 | 1 | 2 | 3 | | 1 | 4 | 3 | | 0 | 0 | 1 | 0 | 0 | 0 | 2 | 1 | 1 | 1 | 0 | 0 | 1 | 2 | 0 | 0 | 0 | 0 | 2 | 0 | 0 |
| 59 | 1 | 4 | 2 | 3 | | 3 | | | 1 | 2 | 2 | 1 | 2 | 2 | | 1 | 1 | 0 | 0 | 2 | 0 | 2 | 1 | 0 | 0 | 1 | 0 | 1 | 1 | 0 | 0 | 1 | 1 | 0 | 1 | 0 |
| 60 | 3 | 1 | 3 | | 1 | 3 | 1 | 1 | 1 | 2 | | 1 | 2 | 2 | | 4 | 1 | 0 | 0 | 0 | 0 | 2 | 2 | 0 | 0 | 2 | 0 | | | 1 | 0 | 1 | 1 | 2 | 1 | 0 |
| 61 | | 2 | 2 | 3 | 2 | 3 | | 1 | | 1 | | 1 | 1 | 2 | | 1 | 1 | 0 | 0 | 2 | 0 | 1 | 1 | 0 | 1 | 0 | 0 | | | 0 | 0 | 0 | 0 | 0 | 0 | 0 |
| 62 | 1 | 4 | 1 | 2 | 1 | 3 | | 1 | | | 1 | 1 | 2 | 1 | | 4 | 3 | 0 | 0 | 1 | 0 | 1 | 0 | 0 | 3 | 1 | 0 | | | 0 | 0 | 0 | 0 | 0 | 1 | 0 |
| 63 | 1 | 1 | 1 | | 1 | 1 | | | 1 | 1 | 1 | | 1 | 2 | | 1 | 0 | 0 | 0 | 2 | 1 | 0 | 0 | 1 | 2 | 2 | 0 | | | 0 | 0 | 1 | 0 | 0 | 0 | 0 |
| 64 | 6 | 3 | 5 | 2 | 2 | 4 | | 2 | | 2 | 1 | 2 | 2 | 1 | 1 | 1 | 1 | | | 2 | | | | | 1 | | | 2 | | | | | | | | |
| 65 | 5 | 5 | 4 | 2 | 2 | 4 | 1 | | | 1 | | 1 | 1 | 4 | | 2 | 3 | 0 | 0 | 0 | 0 | 2 | 2 | 0 | 3 | 1 | 0 | 1 | | 1 | 0 | 0 | 0 | 0 | 0 | 0 |
| 66 | 2 | 2 | | | 1 | | 1 | 1 | | 1 | 1 | | 1 | 1 | | 0 | 0 | 1 | 0 | 1 | 0 | 1 | 1 | 0 | 1 | 0 | 0 | | | 0 | 0 | 1 | 0 | 1 | 0 | 0 |
| 67 | 1 | 1 | 2 | 1 | 2 | 1 | | | | | 1 | 1 | 2 | 2 | | 2 | 1 | 0 | 0 | 2 | 1 | 1 | 1 | 0 | 1 | 0 | 0 | | | 1 | 0 | 0 | 1 | 1 | 0 | 0 |
| 68 | 6 | 5 | 3 | 8 | 5 | 5 | 2 | 2 | 2 | 5 | 3 | 6 | 2 | 1 | | 1 | | | | 1 | | | | | 3 | | | 2 | 2 | | | | | 1 | 1 | |
| 69 | 2 | 3 | 2 | 1 | | | 1 | | 1 | 1 | | 1 | 1 | 2 | | 0 | 0 | 0 | 0 | 0 | 0 | 2 | 2 | 1 | 1 | 1 | 0 | | 1 | 2 | 0 | 0 | 0 | 0 | 0 | 0 |
| 70 | 2 | 1 | 1 | | 1 | | 1 | | | 1 | 2 | 2 | 0 | 2 | | 1 | 1 | 0 | 0 | 1 | 0 | 2 | 2 | 0 | 1 | 0 | 0 | 1 | 1 | 1 | 0 | 0 | 0 | 1 | 0 | 0 |
| 71 | 4 | 2 | 3 | 2 | | 1 | | 1 | 1 | 1 | 2 | 2 | 2 | 0 | | 1 | 0 | 0 | 0 | 2 | 0 | 1 | 1 | 0 | 0 | 1 | 0 | | | 0 | 0 | 1 | 1 | 2 | 0 | 0 |
| 72 | | | | | | 1 | | | | 1 | | | 1 | 0 | | 0 | 1 | 1 | 0 | 1 | 0 | 1 | 1 | 1 | 1 | 1 | 1 | | 1 | 1 | 0 | 0 | 0 | 1 | 0 | 0 |
| 73 | 4 | 3 | 2 | 5 | 2 | 4 | | | | 2 | | | 2 | 1 | | 1 | 1 | 0 | 1 | 3 | 0 | 0 | 0 | 0 | 1 | 1 | 1 | | | 0 | 0 | 0 | 1 | 0 | 0 | 0 |
| 74 | 3 | 1 | 1 | 1 | | 1 | | | | 1 | 1 | 1 | 1 | 1 | | 1 | 0 | 0 | 0 | 1 | 0 | 1 | 0 | 0 | 1 | 1 | 0 | | 1 | 0 | 0 | 0 | 0 | 0 | 0 | 0 |
| 75 | 1 | 1 | | 1 | | 3 | | 1 | | 2 | | 2 | 0 | 1 | | 1 | 1 | 0 | 0 | 3 | 0 | 2 | 2 | 1 | 0 | 0 | 0 | 1 | | 0 | 0 | 0 | 0 | 0 | 1 | 0 |
| 76 | 24 | 10 | 7 | 5 | 13 | 12 | 1 | 2 | | 1 | | | 3 | 4 | | 3 | 5 | 0 | 0 | 2 | 0 | 0 | 0 | 0 | 1 | 1 | 0 | 1 | | 0 | 0 | 1 | 1 | 0 | 0 | 0 |
| 77 | 13 | 6 | 2 | | 5 | 6 | 3 | 2 | 1 | | 1 | 2 | 1 | 0 | | 0 | 2 | 0 | 0 | 1 | 0 | 0 | 1 | 1 | 0 | 0 | 0 | | 1 | 0 | 1 | 0 | 0 | 0 | 0 | 0 |
| 78 | 1 | 1 | 1 | | | | | | | | 1 | | 0 | 0 | | 1 | 1 | 2 | 1 | 1 | 1 | 1 | 0 | 1 | 1 | 0 | 0 | | | 0 | 1 | 0 | 0 | 0 | 0 | 0 |
| 79 | 3 | 3 | 2 | 3 | 5 | 5 | | 1 | 2 | 3 | | 1 | 0 | 2 | | 0 | 0 | 1 | 0 | 1 | 2 | 0 | 1 | 0 | 2 | 0 | 0 | 1 | 1 | 1 | 0 | 0 | 0 | 0 | 0 | 0 |
| 80 | 3 | 7 | 5 | 4 | 3 | 6 | 1 | | | 3 | 2 | 4 | 0 | 2 | | 1 | 0 | 0 | 0 | 1 | 0 | 1 | 1 | 0 | 0 | 1 | 0 | | | 1 | 0 | 1 | 0 | 1 | 1 | 0 |
| 81 | 1 | 6 | 4 | 2 | | 5 | 1 | 1 | | 1 | | | 0 | 2 | | 1 | 1 | 0 | 0 | 0 | 0 | 0 | 1 | 1 | 1 | 0 | 1 | | 1 | 0 | 0 | 0 | 1 | 0 | 1 | 0 |
| 82 | 2 | 2 | 1 | 1 | 1 | 1 | | | | 3 | 2 | 1 | 2 | 1 | | 1 | 1 | 0 | 0 | 1 | 0 | 0 | 1 | 0 | 0 | 0 | 0 | | | 0 | 0 | 0 | 0 | 0 | 0 | 0 |
| 83 | 2 | | 1 | | 1 | 1 | | | | 1 | 1 | 1 | 3 | 0 | 1 | 1 | 0 | 0 | 0 | 0 | 0 | 2 | 1 | 1 | 0 | 0 | 0 | | | 0 | 0 | 0 | 0 | 0 | 0 | 0 |
| 84 | 1 | 2 | 2 | | | 1 | | | | 1 | | | 2 | 2 | | 1 | 1 | 0 | 1 | 2 | 0 | 1 | 1 | 0 | 0 | 0 | 0 | | | 0 | 0 | 0 | 0 | 0 | 0 | 0 |
| 85 | 1 | 1 | | 1 | 1 | 1 | | | | | 1 | | 0 | 1 | | 0 | 1 | 1 | 1 | 0 | 0 | 1 | 0 | 1 | 2 | 1 | 0 | | | 0 | 0 | 0 | 0 | 0 | 0 | 0 |
| 86 | 7 | 4 | 1 | | 3 | 2 | | 3 | 1 | 2 | 1 | 1 | 7 | 1 | | 0 | 5 | 4 | 0 | 0 | 0 | 0 | 0 | 0 | 0 | 0 | 0 | | 1 | 0 | 0 | 0 | 0 | 0 | 0 | 0 |
| 87 | 2 | 1 | | | 1 | 1 | | | | 1 | 1 | 2 | 1 | 0 | | 1 | 1 | 0 | 0 | 1 | 0 | 1 | 0 | 0 | 1 | 0 | 0 | | | 1 | 0 | 0 | 0 | 0 | 0 | 0 |
| 88 | 1 | 1 | 2 | | 2 | | 1 | 1 | 1 | 1 | | | 1 | 1 | | 0 | 1 | 0 | 0 | 1 | 0 | 0 | 0 | 0 | 0 | 0 | 1 | | 1 | 0 | 0 | 0 | 0 | 0 | 0 | 0 |
| 89 | 2 | 2 | 1 | 1 | 1 | | | | | | | | 1 | 1 | | 1 | 1 | 0 | 0 | 1 | 0 | 1 | 1 | 1 | 1 | 0 | 0 | | | 0 | 0 | 1 | 0 | 0 | 0 | 0 |
| 90 | 1 | 2 | 2 | 1 | 2 | 2 | | | | 1 | | 1 | 1 | 3 | | 2 | 3 | 1 | 0 | 4 | 0 | 0 | 0 | 0 | 0 | 1 | 0 | 1 | | 0 | 1 | 0 | 0 | 0 | 0 | 0 |
| 91 | 3 | 2 | 2 | 1 | 3 | 3 | | | | 1 | | | 1 | 2 | 1 | 2 | 2 | | | 2 | | | | | 1 | 1 | | | | | | | | 1 | 1 | |
| 92 | 3 | 2 | 1 | 1 | | 1 | | 2 | 1 | 2 | 1 | | 1 | 0 | | 0 | 1 | 1 | 0 | 0 | 0 | 1 | 0 | 0 | 0 | 0 | 1 | | 1 | 0 | 0 | 0 | 1 | 0 | 0 | 0 |
| 93 | 1 | 2 | 2 | 2 | | 2 | 1 | 1 | | 2 | 1 | 1 | 2 | 1 | | 0 | 0 | 0 | 0 | 0 | 0 | 1 | 0 | 0 | 0 | 1 | 0 | | | 1 | 0 | 0 | 0 | 1 | 0 | 0 |
| 94 | | 3 | | | | 2 | | 1 | 1 | | 1 | 1 | 0 | 0 | | 0 | 0 | 0 | 1 | 2 | 0 | 0 | 2 | 0 | 1 | 1 | 0 | | | 1 | 0 | 0 | 0 | 0 | 0 | 0 |
| 95 | | | | 2 | | 1 | | | | 1 | | 1 | 1 | 2 | | 1 | 1 | 0 | 0 | 1 | 0 | 1 | 0 | 0 | 2 | 0 | 0 | | | 0 | 0 | 1 | 1 | 0 | 0 | 0 |
| 96 | 1 | | 4 | | 2 | 3 | 1 | 1 | 1 | 1 | 1 | | 0 | 1 | | 1 | 0 | 0 | 0 | 0 | 0 | 1 | 2 | 0 | 0 | 0 | 0 | | | 0 | 0 | 0 | 1 | 1 | 0 | 0 |
| 97 | | | | | | | | | | | | | 0 | 0 | | 1 | 0 | 1 | 1 | 1 | 1 | 0 | 1 | 1 | 1 | 0 | 1 | | | 0 | 1 | 1 | 0 | 0 | 0 | 0 |
| 98 | 1 | 7 | 3 | 2 | 3 | 6 | | 1 | | 1 | 1 | 3 | 0 | 3 | 1 | 0 | 0 | 0 | 1 | 0 | 0 | 0 | 5 | 0 | 3 | 0 | 0 | 1 | | 1 | 0 | 1 | 1 | 1 | 0 | 0 |
| 99 | 5 | 4 | 4 | 4 | 1 | 2 | 4 | 1 | | 2 | 2 | 2 | | 1 | | 1 | | | | | | | | | 1 | | | | | | | | | | | |
| 100 | 7 | 4 | 6 | | 1 | 3 | | | | | 1 | | 1 | 1 | | 4 | 1 | 0 | 1 | 0 | 0 | 1 | 6 | 0 | 0 | 0 | 0 | | | 0 | 0 | 0 | 0 | 0 | 0 | 0 |
| 101 | | 7 | 1 | | 1 | 10 | 1 | | 1 | 3 | 1 | | 6 | 0 | | 0 | 0 | 0 | 0 | 2 | 2 | 0 | 0 | 0 | 0 | 1 | 0 | | | 0 | 0 | 0 | 0 | 0 | 0 | 0 |
| 102 | 2 | 5 | 1 | | 5 | 4 | | | | | | | 3 | 3 | | 1 | 0 | 0 | 1 | 2 | 1 | 0 | 0 | 0 | 2 | 1 | 0 | | | 0 | 0 | 0 | 0 | 0 | 0 | 0 |
| 103 | 1 | 1 | 1 | 1 | | | | | 1 | | | | 0 | 0 | | 1 | 1 | 0 | 0 | 1 | 0 | 1 | 1 | 1 | 0 | 1 | 0 | | | 0 | 0 | 0 | 0 | 0 | 0 | 0 |
| 104 | 1 | | 1 | | | | | | | | | | 0 | 1 | | 1 | 1 | 0 | 0 | 1 | 1 | 0 | 0 | 1 | 2 | 2 | 0 | | | 0 | 0 | 0 | 0 | 0 | 0 | 0 |
| 105 | | | | 1 | | | 1 | 1 | 1 | 1 | 1 | 1 | 0 | 0 | | 1 | 0 | 0 | 0 | 0 | 0 | 0 | 0 | 0 | 0 | 1 | 0 | | | 0 | 0 | 0 | 0 | 0 | 0 | 0 |
| 106 | | 1 | | | | | | | | | | | 0 | 1 | | 1 | 0 | 0 | 0 | 2 | 1 | 1 | 1 | 1 | 0 | 1 | 0 | | | 0 | 0 | 0 | 0 | 0 | 0 | 0 |
| 107 | 3 | | 1 | 1 | 1 | 4 | | 1 | | | 1 | 1 | 5 | 0 | | 2 | 4 | 1 | 0 | 0 | 0 | 0 | 3 | 0 | 0 | 0 | 0 | 1 | | 0 | 0 | 0 | 0 | 0 | 0 | 0 |
| 108 | 3 | 1 | 1 | 4 | 2 | 1 | | | 1 | 3 | 1 | 3 | 0 | 0 | | 0 | 0 | 0 | 0 | 1 | 0 | 0 | 1 | 0 | 2 | 1 | 0 | | | 0 | 0 | 0 | 1 | 0 | 0 | 0 |
| 109 | 3 | 2 | 2 | 1 | 1 | 3 | 1 | 2 | | | 5 | 1 | 0 | 0 | | 0 | 0 | 1 | 0 | 0 | 1 | 0 | 0 | 1 | 0 | 1 | 0 | 1 | | 0 | 0 | 0 | 0 | 0 | 0 | 0 |
| 110 | 2 | 5 | 3 | 1 | 2 | 1 | | 2 | 1 | | | 1 | 2 | 2 | | 1 | 0 | 1 | 0 | 0 | 0 | 0 | 0 | 1 | 0 | 0 | 0 | | 1 | 0 | 0 | 0 | 0 | 0 | 0 | 0 |
| 111 | 1 | | 1 | 1 | | | 1 | 1 | 1 | 1 | 2 | 1 | 0 | 0 | | 0 | 2 | 0 | 1 | 0 | 0 | 0 | 0 | 0 | 0 | 0 | 0 | | | 0 | 0 | 0 | 1 | 0 | 0 | 0 |
| 112 | | 1 | 3 | 1 | | | | 1 | | | 1 | 1 | 0 | 0 | | 0 | 0 | 2 | 0 | 1 | 1 | 0 | 1 | 0 | 0 | 1 | 0 | | | 1 | 0 | 0 | 0 | 0 | 0 | 0 |
| 113 | 1 | | | | | | | | | | | | 0 | 1 | 1 | 1 | 1 | 0 | 0 | 2 | 0 | 1 | 1 | 0 | 1 | 0 | 0 | 1 | | 0 | 0 | 0 | 0 | 0 | 0 | 0 |
| 114 | 4 | 3 | 4 | 1 | 2 | 2 | 1 | 1 | | | | 1 | 1 | 0 | | 0 | 1 | 0 | 0 | 0 | 0 | 0 | 1 | 0 | 1 | 1 | 0 | 1 | | 0 | 0 | 0 | 0 | 0 | 0 | 0 |
| 115 | 1 | 1 | 3 | 1 | 1 | 2 | | 2 | | 2 | 2 | 1 | | | | 2 | 3 | | | 1 | | | | | 1 | | | | 1 | | | | 1 | | | |
| 116 | | 2 | | 2 | 1 | 1 | | | | | | | 0 | 0 | | 2 | 1 | 0 | 1 | 2 | 0 | 1 | 2 | 0 | 2 | 1 | 0 | | | 1 | 0 | 0 | 0 | 0 | 0 | 0 |
| 117 | 3 | 2 | 3 | 4 | 3 | 3 | | | | 1 | | | 0 | 1 | 1 | 1 | 2 | 0 | 0 | 2 | 0 | 1 | 2 | 0 | 0 | 0 | 0 | 1 | | 0 | 0 | 0 | 1 | 0 | 0 | 0 |
| 118 | 2 | 1 | 2 | | | 2 | | 1 | | 1 | | | 1 | 2 | | 3 | 4 | 1 | 0 | 2 | 0 | 0 | 0 | 0 | 0 | 0 | 0 | | 1 | 1 | 0 | 0 | 0 | 0 | 1 | 0 |
| 119 | | 2 | 4 | | | 1 | | | | 2 | | | 1 | 1 | | 1 | 2 | 0 | 0 | 4 | 0 | 1 | 2 | 0 | 0 | 0 | 0 | 2 | | 1 | 0 | 0 | 0 | 0 | 0 | 0 |
| 120 | 4 | 8 | 4 | 3 | 1 | 4 | 1 | 1 | 1 | 2 | 1 | 1 | 0 | 1 | | 0 | 0 | 0 | 0 | 0 | 0 | 1 | 0 | 0 | 0 | 0 | 0 | 1 | 1 | 0 | 0 | 0 | 0 | 0 | 0 | 0 |
| 121 | 12 | 10 | 10 | 8 | 11 | 9 | | | | | | | 0 | 4 | | 10 | 7 | 0 | 0 | 7 | 0 | 2 | 2 | 0 | 1 | 1 | 0 | | | 1 | 0 | 4 | 2 | 2 | 0 | 0 |
| 122 | 2 | 4 | 3 | 4 | 3 | 2 | | | | | | | 0 | 1 | | 3 | 2 | 0 | 0 | 1 | 1 | 0 | 0 | 0 | 1 | 3 | 0 | | | 0 | 0 | 0 | 0 | 0 | 0 | 0 |
| 123 | 2 | 1 | 1 | 1 | 1 | 2 | 2 | | 1 | 1 | 1 | 1 | 0 | 1 | | 0 | 0 | 0 | 0 | 0 | 0 | 0 | 0 | 0 | 0 | 1 | 0 | | | 0 | 0 | 0 | 0 | 0 | 0 | 0 |
| 124 | 2 | 2 | 2 | | | 2 | 1 | | | 1 | 1 | 2 | 0 | 0 | | 1 | 1 | 0 | 0 | 0 | 0 | 0 | 1 | 0 | 0 | 0 | 0 | | | 0 | 0 | 0 | 0 | 0 | 0 | 0 |
| 125 | 1 | 1 | | | 1 | 2 | | | | | | 1 | 1 | 1 | | 0 | 1 | 1 | 0 | 2 | 0 | 0 | 0 | 1 | 0 | 0 | 0 | | | 0 | 0 | 0 | 0 | 0 | 0 | 0 |
| 126 | | | | 2 | | 1 | 1 | | | 1 | 1 | | 1 | 0 | | 1 | 1 | 0 | 0 | 0 | 0 | 0 | 0 | 0 | 1 | 0 | 0 | | | 0 | 0 | 0 | 0 | 0 | 0 | 0 |
| 127 | 2 | 2 | | 3 | | | | 1 | | | 3 | 1 | 3 | 0 | | 1 | 0 | 0 | 0 | 0 | 0 | 1 | 2 | 0 | 0 | 0 | 0 | | | 0 | 0 | 0 | 0 | 0 | 0 | 0 |
| 128 | | 1 | 2 | 2 | 2 | | 1 | | | 2 | 1 | 1 | 0 | 0 | | 0 | 0 | 0 | 0 | 0 | 0 | 1 | 1 | 3 | 0 | 0 | 0 | | | 0 | 0 | 0 | 0 | 0 | 0 | 0 |
| 129 | 2 | 1 | 1 | | | 1 | | | | 1 | 1 | | 0 | 1 | | 0 | 0 | 0 | 0 | 0 | 0 | 1 | 1 | 0 | 0 | 1 | 1 | | 1 | 0 | 0 | 0 | 0 | 0 | 0 | 0 |
| 130 | 1 | 1 | | 1 | | | | | | | | 1 | 0 | 1 | | 0 | 1 | 0 | 0 | 1 | 0 | 1 | 1 | 1 | 0 | 0 | 0 | | | 0 | 0 | 0 | 0 | 0 | 1 | 0 |
| 131 | | 1 | 2 | 1 | | 1 | | 1 | | 1 | 1 | 1 | 0 | 0 | | 1 | 0 | 0 | 0 | 0 | 0 | 1 | 0 | 0 | 1 | 0 | 0 | | | 0 | 0 | 0 | 1 | 0 | 0 | 0 |
| 132 | | 1 | 1 | 1 | | | 1 | | | | 1 | 1 | 1 | 0 | | 0 | 0 | 1 | 0 | 0 | 1 | 0 | 0 | 1 | 0 | 0 | 0 | 1 | | 0 | 0 | 0 | 0 | 0 | 0 | 0 |
| 122 | 1 | 1 | | | 1 | | 1 | 1 | 1 | 1 | | 1 | 0 | 1 | | 0 | 0 | 0 | 0 | 0 | 0 | 0 | 0 | 0 | 0 | 1 | 0 | | | 0 | 1 | 0 | 0 | 0 | 0 | 0 |
| 134 | | 1 | | 1 | | | 1 | | | | | | 0 | 1 | | 0 | 0 | 1 | 0 | 0 | 1 | 1 | 1 | 0 | 1 | 0 | 0 | | | 0 | 0 | 0 | 0 | 0 | 0 | 0 |
| 135 | | | | | | | | | | | | | 0 | 1 | | 0 | 0 | 0 | 0 | 1 | 0 | 1 | 1 | 1 | 1 | 1 | 0 | 1 | | 0 | 0 | 0 | 0 | 0 | 0 | 0 |
| 136 | 3 | 1 | 1 | 1 | 1 | 1 | | | 1 | 1 | 2 | | 2 | 0 | | 0 | 1 | 0 | 0 | 0 | 0 | 0 | 2 | 1 | 0 | 0 | 0 | | 1 | 0 | 0 | 0 | 0 | 0 | 0 | 0 |
| 137 | 1 | 2 | | 1 | 1 | 1 | | | | | | | 2 | 0 | | 2 | 0 | 0 | 0 | 1 | 1 | 1 | 1 | 1 | 0 | 0 | 0 | | | 0 | 0 | 1 | 0 | 0 | 0 | 0 |
| 138 | 2 | 1 | 2 | 1 | 1 | 1 | | 1 | | | | | 1 | 1 | | 1 | 0 | 1 | 0 | 0 | 0 | 0 | 1 | 0 | 0 | 2 | 0 | | | 0 | 0 | 1 | 1 | 0 | 0 | 0 |
| 139 | | | 2 | | | 1 | | | | | 1 | 1 | 0 | 1 | | 0 | 0 | 0 | 1 | 1 | 0 | 0 | 0 | 0 | 0 | 1 | 1 | | | 1 | 0 | 1 | 0 | 0 | 0 | 0 |
| 140 | | | | | 1 | | 1 | | | | | | 1 | 0 | | 1 | 1 | 1 | 0 | 0 | 0 | 1 | 0 | 1 | 0 | 0 | 0 | | | 0 | 0 | 0 | 1 | 1 | 0 | 0 |

| | Column | | | | | | | | | |
|---|---|---|---|---|---|---|---|---|---|---|
| | | | | | | 51 | 52 | | | |
| | | | | 49 | | [symbol] | [symbol] | | 54 | 55 |
| | 46 | 47 | 48 | # | 50 | Peptides | Peptides | | % | % |
| | Sample# | | | Peptides | # Peptides in | per | per | 53 | Cancers Expressing | Normals Expressing |
| Row | 43 | 44 | 45 | in Cancer | Normal | Cancer Sample | Normal Sample | Ratio | Protein | Protein |
| 1 | 0 | | 3 | 115 | 13 | 3.5 | 1.1 | 3.2 | 100% | 50% |
| 2 | 0 | | 3 | 194 | 25 | 5.9 | 2.1 | 2.8 | 100% | 75% |
| 3 | 2 | | 2 | 158 | 22 | 4.8 | 1.8 | 2.6 | 100% | 92% |
| 4 | 2 | | 0 | 156 | 13 | 4.7 | 1.1 | 4.4 | 95% | 67% |
| 5 | 2 | | 0 | 95 | 15 | 2.9 | 1.3 | 2.3 | 95% | 75% |
| 6 | 0 | | 0 | 182 | 9 | 5.5 | 0.8 | 7.4 | 90% | 50% |
| 7 | 0 | 1 | 1 | 138 | 18 | 4.2 | 1.5 | 2.8 | 90% | 75% |
| 8 | 2 | | 0 | 88 | 13 | 2.7 | 1.1 | 2.5 | 90% | 67% |
| 9 | 0 | | 3 | 119 | 21 | 3.6 | 1.8 | 2.1 | 90% | 75% |
| 10 | 1 | | 0 | 137 | 10 | 4.2 | 0.8 | 5.0 | 86% | 42% |
| 11 | 0 | | 0 | 124 | 6 | 3.8 | 0.5 | 7.5 | 81% | 42% |
| 12 | 1 | | 0 | 250 | 13 | 7.6 | 1.1 | 7.0 | 81% | 50% |
| 13 | 0 | | 0 | 74 | 7 | 2.2 | 0.6 | 3.8 | 81% | 42% |
| 14 | 0 | | 1 | 114 | 12 | 3.5 | 1.0 | 3.5 | 81% | 58% |
| 15 | 0 | | 0 | 47 | 4 | 1.4 | 0.3 | 4.3 | 76% | 33% |
| 16 | 1 | | 0 | 202 | 16 | 6.1 | 1.3 | 4.6 | 76% | 50% |
| 17 | 0 | | 0 | 26 | 5 | 0.8 | 0.4 | 1.9 | 76% | 42% |
| 18 | 0 | | 0 | 36 | 1 | 1.1 | 0.1 | 13.1 | 71% | 8% |
| 19 | 0 | | 0 | 72 | 3 | 2.2 | 0.3 | 8.7 | 71% | 25% |
| 20 | 0 | | 0 | 84 | 6 | 2.5 | 0.5 | 5.1 | 71% | 42% |
| 21 | 0 | | 0 | 59 | 5 | 1.8 | 0.4 | 4.3 | 71% | 42% |
| 22 | 0 | | 0 | 187 | 8 | 5.7 | 0.7 | 8.5 | 71% | 42% |
| 23 | 0 | | 0 | 89 | 9 | 2.7 | 0.8 | 3.6 | 71% | 50% |
| 24 | 0 | | 2 | 100 | 15 | 3.0 | 1.3 | 2.4 | 71% | 67% |
| 25 | 1 | | 0 | 183 | 18 | 5.5 | 1.5 | 3.7 | 71% | 58% |
| 26 | 0 | | 0 | 35 | 0 | 1.1 | 0.0 | NA | 67% | 0% |
| 27 | 0 | | 0 | 95 | 0 | 2.9 | 0.0 | NA | 67% | 0% |
| 28 | 0 | | 0 | 47 | 1 | 1.4 | 0.1 | 17.1 | 67% | 8% |
| 29 | 1 | | 0 | 33 | 1 | 1.0 | 0.1 | 12.0 | 67% | 8% |
| 30 | 0 | | 0 | 35 | 2 | 1.1 | 0.2 | 6.4 | 67% | 17% |
| 31 | 0 | | 0 | 54 | 4 | 1.6 | 0.3 | 4.9 | 67% | 25% |
| 32 | 0 | | 0 | 34 | 3 | 1.0 | 0.3 | 4.1 | 67% | 17% |
| 33 | 0 | | 1 | 83 | 5 | 2.5 | 0.4 | 6.0 | 67% | 42% |
| 34 | | | | 99 | 6 | 3.0 | 0.5 | 6.0 | 67% | 42% |
| 35 | 0 | | 0 | 45 | 4 | 1.4 | 0.3 | 4.1 | 67% | 25% |
| 36 | 1 | | 0 | 32 | 4 | 1.0 | 0.3 | 2.9 | 67% | 33% |
| 37 | 0 | | 0 | 79 | 6 | 2.4 | 0.5 | 4.8 | 67% | 50% |
| 38 | 2 | | 0 | 47 | 7 | 1.4 | 0.6 | 2.4 | 67% | 50% |
| 39 | 0 | | 0 | 41 | 6 | 1.2 | 0.5 | 2.5 | 67% | 42% |
| 40 | 0 | | 0 | 65 | 0 | 2.0 | 0.0 | NA | 62% | 0% |
| 41 | 0 | | 0 | 112 | 2 | 3.4 | 0.2 | 20.4 | 62% | 17% |
| 42 | 0 | | 0 | 24 | 2 | 0.7 | 0.2 | 4.4 | 62% | 17% |
| 43 | 0 | | 0 | 41 | 3 | 1.2 | 0.3 | 5.0 | 62% | 8% |
| 44 | 1 | | 0 | 89 | 7 | 2.7 | 0.6 | 4.6 | 62% | 50% |
| 45 | | | | 36 | 3 | 1.1 | 0.3 | 4.4 | 62% | 25% |
| 46 | 0 | | 0 | 51 | 5 | 1.5 | 0.4 | 3.7 | 62% | 33% |
| 47 | 0 | | 0 | 38 | 1 | 1.2 | 0.1 | 13.8 | 57% | 8% |
| 48 | 0 | | 0 | 25 | 1 | 0.8 | 0.1 | 9.1 | 57% | 8% |
| 49 | 0 | | 0 | 54 | 2 | 1.6 | 0.2 | 9.8 | 57% | 17% |
| 50 | 0 | | 0 | 52 | 3 | 1.6 | 0.3 | 6.3 | 57% | 17% |
| 51 | 0 | | 0 | 129 | 5 | 3.9 | 0.4 | 9.4 | 57% | 25% |
| 52 | 0 | | 0 | 88 | 5 | 2.7 | 0.4 | 6.4 | 57% | 33% |
| 53 | 0 | | 0 | 82 | 3 | 2.5 | 0.3 | 9.9 | 57% | 25% |
| 54 | 1 | | 1 | 54 | 3 | 1.6 | 0.3 | 6.5 | 57% | 25% |
| 55 | 0 | | 0 | 58 | 3 | 1.8 | 0.3 | 7.0 | 57% | 17% |
| 65 | 0 | | 0 | 51 | 4 | 1.5 | 0.3 | 4.6 | 57% | 25% |
| 57 | 1 | | 0 | 18 | 3 | 0.5 | 0.3 | 2.2 | 57% | 25% |
| 58 | 0 | | 0 | 92 | 5 | 2.8 | 0.4 | 6.7 | 57% | 25% |
| 59 | 0 | | 0 | 48 | 5 | 1.5 | 0.4 | 3.5 | 57% | 42% |
| 60 | 0 | | 0 | 50 | 6 | 1.5 | 0.5 | 3.0 | 57% | 42% |
| 61 | 0 | | 0 | 33 | 0 | 1.0 | 0.0 | NA | 52% | 0% |
| 62 | 0 | | 0 | 33 | 1 | 1.0 | 0.1 | 12.0 | 52% | 8% |
| 63 | 0 | | 0 | 25 | 1 | 0.8 | 0.1 | 9.1 | 52% | 8% |
| 64 | | | | 61 | 2 | 1.8 | 0.2 | 11.1 | 52% | 8% |
| 65 | 0 | | 1 | 62 | 3 | 1.9 | 0.3 | 7.5 | 52% | 25% |
| 66 | 0 | | 0 | 24 | 2 | 0.7 | 0.2 | 4.4 | 52% | 17% |
| 67 | 0 | | 0 | 32 | 3 | 1.0 | 0.3 | 3.9 | 52% | 25% |
| 68 | | | | 89 | 6 | 2.7 | 0.5 | 5.4 | 52% | 33% |
| 69 | 0 | | 0 | 30 | 3 | 0.9 | 0.3 | 3.6 | 52% | 17% |
| 70 | 0 | | 0 | 24 | 4 | 0.7 | 0.3 | 2.2 | 52% | 33% |
| 71 | 0 | | 0 | 42 | 4 | 1.3 | 0.3 | 3.8 | 52% | 25% |
| 72 | 0 | | 0 | 16 | 3 | 0.5 | 0.3 | 1.9 | 52% | 25% |
| 73 | 0 | | 0 | 41 | 1 | 1.2 | 0.1 | 14.9 | 48% | 8% |
| 74 | 0 | | 0 | 27 | 1 | 0.8 | 0.1 | 9.8 | 48% | 8% |
| 75 | 0 | | 0 | 37 | 2 | 1.1 | 0.2 | 6.7 | 48% | 17% |
| 76 | 0 | | 0 | 163 | 3 | 4.9 | 0.3 | 19.8 | 48% | 25% |
| 77 | 0 | | 0 | 85 | 2 | 2.6 | 0.2 | 15.5 | 48% | 17% |
| 78 | 0 | | 1 | 17 | 2 | 0.5 | 0.2 | 3.1 | 48% | 17% |
| 79 | 0 | | 0 | 53 | 3 | 1.6 | 0.3 | 6.4 | 48% | 25% |
| 80 | 0 | | 0 | 78 | 4 | 2.4 | 0.3 | 7.1 | 48% | 33% |
| 81 | 0 | | 0 | 48 | 3 | 1.5 | 0.3 | 5.8 | 48% | 25% |
| 82 | 0 | | 0 | 38 | 0 | 1.2 | 0.0 | NA | 43% | 0% |
| 83 | 0 | | 0 | 27 | 0 | 0.8 | 0.0 | NA | 43% | 0% |
| 84 | 0 | | 0 | 28 | 0 | 0.8 | 0.0 | NA | 43% | 0% |
| 85 | 0 | | 0 | 16 | 0 | 0.5 | 0.0 | NA | 43% | 0% |
| 86 | 0 | | 0 | 61 | 1 | 1.8 | 0.1 | 22.2 | 43% | 8% |
| 87 | 0 | | 0 | 26 | 1 | 0.8 | 0.1 | 9.5 | 43% | 8% |
| 88 | 0 | | 0 | 27 | 1 | 0.8 | 0.1 | 9.8 | 43% | 8% |
| 89 | 0 | | 0 | 19 | 1 | 0.6 | 0.1 | 6.9 | 43% | 8% |
| 90 | 0 | | 0 | 30 | 2 | 0.9 | 0.2 | 5.5 | 43% | 17% |
| 91 | | | | 38 | 2 | 1.2 | 0.2 | 6.9 | 43% | 17% |
| 92 | 0 | | 0 | 32 | 2 | 1.0 | 0.2 | 5.8 | 43% | 17% |
| 93 | 0 | | 0 | 25 | 2 | 0.8 | 0.2 | 4.5 | 43% | 17% |
| 94 | 1 | | 0 | 23 | 2 | 0.7 | 0.2 | 4.2 | 43% | 17% |
| 95 | 0 | | 0 | 17 | 2 | 0.5 | 0.2 | 3.1 | 43% | 17% |
| 96 | 0 | | 0 | 28 | 2 | 0.8 | 0.2 | 5.1 | 43% | 17% |
| 97 | 0 | | 0 | 10 | 2 | 0.3 | 0.2 | 1.8 | 43% | 17% |
| 98 | 0 | | 0 | 54 | 5 | 1.6 | 0.4 | 3.9 | 43% | 42% |
| 99 | | | | 65 | 0 | 2.0 | 0.0 | NA | 38% | 0% |
| 100 | 0 | | 0 | 57 | 0 | 1.7 | 0.0 | NA | 38% | 0% |
| 101 | 0 | | 0 | 72 | 0 | 2.2 | 0.0 | NA | 38% | 0% |
| 102 | 0 | | 0 | 37 | 0 | 1.1 | 0.0 | NA | 38% | 0% |
| 103 | 0 | | 0 | 18 | 0 | 0.5 | 0.0 | NA | 38% | 0% |
| 104 | 0 | | 0 | 16 | 0 | 0.5 | 0.0 | NA | 38% | 0% |
| 105 | 0 | | 0 | 10 | 0 | 0.3 | 0.0 | NA | 38% | 0% |
| 106 | 0 | | 0 | 10 | 0 | 0.3 | 0.0 | NA | 38% | 0% |
| 107 | 0 | | 0 | 37 | 1 | 1.1 | 0.1 | 13.5 | 38% | 8% |
| 108 | 0 | | 0 | 43 | 1 | 1.3 | 0.1 | 15.6 | 38% | 8% |
| 109 | 0 | | 0 | 27 | 1 | 0.8 | 0.1 | 9.8 | 38% | 8% |
| 110 | 0 | | 0 | 41 | 1 | 1.2 | 0.1 | 14.9 | 38% | 8% |
| 111 | 0 | | 0 | 23 | 1 | 0.7 | 0.1 | 8.4 | 38% | 8% |
| 112 | 0 | | 0 | 19 | 1 | 0.6 | 0.1 | 6.9 | 38% | 8% |
| 113 | 0 | | 0 | 10 | 1 | 0.3 | 0.1 | 3.6 | 38% | 8% |
| 114 | 0 | | 0 | 39 | 1 | 1.2 | 0.1 | 14.2 | 38% | 8% |
| 115 | | | | 43 | 2 | 1.3 | 0.2 | 7.8 | 38% | 17% |
| 116 | 0 | | 1 | 24 | 2 | 0.7 | 0.2 | 4.4 | 38% | 17% |
| 117 | 0 | | 0 | 36 | 2 | 1.1 | 0.2 | 6.5 | 38% | 17% |
| 118 | 0 | | 0 | 31 | 3 | 0.9 | 0.3 | 3.8 | 38% | 25% |
| 119 | 0 | | 0 | 34 | 3 | 1.0 | 0.3 | 4.1 | 38% | 17% |
| 120 | 0 | | 0 | 63 | 2 | 1.9 | 0.2 | 11.5 | 38% | 17% |
| 121 | 0 | | 0 | 136 | 9 | 4.1 | 0.8 | 5.5 | 38% | 33% |
| 122 | 0 | | 0 | 49 | 0 | 1.5 | 0.0 | NA | 33% | 0% |
| 123 | 0 | | 0 | 25 | 0 | 0.8 | 0.0 | NA | 33% | 0% |
| 124 | 0 | | 0 | 23 | 0 | 0.7 | 0.0 | NA | 33% | 0% |
| 125 | 0 | | 0 | 20 | 0 | 0.6 | 0.0 | NA | 33% | 0% |
| 126 | 0 | | 0 | 17 | 0 | 0.5 | 0.0 | NA | 33% | 0% |
| 127 | 1 | | 0 | 30 | 1 | 0.9 | 0.1 | 10.9 | 33% | 8% |
| 128 | 0 | | 1 | 27 | 1 | 0.8 | 0.1 | 9.8 | 33% | 8% |
| 129 | 0 | | 0 | 17 | 1 | 0.5 | 0.1 | 6.2 | 33% | 8% |
| 130 | 0 | | 0 | 14 | 1 | 0.4 | 0.1 | 5.1 | 33% | 8% |
| 131 | 0 | | 0 | 20 | 1 | 0.6 | 0.1 | 7.3 | 33% | 8% |
| 132 | 0 | | 0 | 12 | 1 | 0.4 | 0.1 | 4.4 | 33% | 8% |
| 122 | 0 | | 0 | 11 | 1 | 0.3 | 0.1 | 4.0 | 33% | 8% |
| 134 | 1 | | 0 | 10 | 1 | 0.3 | 0.1 | 3.6 | 33% | 8% |
| 135 | 0 | | 0 | 8 | 1 | 0.2 | 0.1 | 2.9 | 33% | 8% |
| 136 | 0 | | 1 | 30 | 2 | 0.9 | 0.2 | 5.5 | 33% | 17% |
| 137 | 1 | | 0 | 19 | 2 | 0.6 | 0.2 | 3.5 | 33% | 17% |
| 138 | 0 | | 0 | 21 | 2 | 0.6 | 0.2 | 3.8 | 33% | 17% |
| 139 | 0 | | 0 | 12 | 2 | 0.4 | 0.2 | 2.2 | 33% | 17% |
| 140 | 0 | | 0 | 8 | 2 | 0.2 | 0.2 | 1.5 | 33% | 17% |

[symbol]

indicates data missing or illegible when filed

For example, the top protein listed (Elongation factor 2) was found to be expressed in 33/33 cancer samples and 6/12 normal samples, and where the total number of unique peptide hits for this protein across all cancer samples was 115, while the total number of unique peptide hits across all normal samples was 13. The ratio of 3.5 is based on the average number of peptides from this protein in the cancer samples vs. the normal samples. Thus, based on this increased ratio in expression levels across a very high percentage of cancer samples, this protein is considered a biomarker of early stage endometrial cancer. Based on the exact same data analysis across greater than 13,000 unique proteins, the list of proteins in Table 1 was selected as those proteins that can best diagnose, prognose, and provide for novel therapeutic targets.

This list was further analyzed to determine those proteins that represent the best biomarkers for early stage endometrial cancer. Table 2, in which samples entries 1-33 are for the cancer samples and 34-45 for the normal samples, shows the same data analysis on the most significant 12 proteins from the proteins in Table 1. These 12 proteins are considered the most significant biomarkers based on their overall increased expression levels in cancer vs. normal and the high percentage of expression across all the cancer samples. Each of these 12 proteins demonstrates at least a 3.8 fold increased expression in cancer over normal and where a large percentage of the cancer samples are expressing each of these proteins (>67%). The top 4 listed proteins (GSTP-I, Transgelin-2, 6PGD, and Vinculin) all show large increases in expression in cancer over normal and are expressed in at least 80% of all the cancer samples.

The biological function of the proteins in Table 2 was identified by a literature search and each of the supporting references for each protein is listed in Table 3, which is attached hereto and incorporated herein in its entirety. There is no apparent bias towards anyone particular type of protein based on function in this collection of 12 candidate biomarkers.

TABLE 2

| | Column | | | | | | | | | | |
|---|---|---|---|---|---|---|---|---|---|---|---|
| | | | | 4 | 5 | 6 | 7 | 8 | 9 | 10 | 11 |
| | 1 | 2 | 3 | Sample # | | | | | | | |
| Row | Protein Name | Reference | Accession | 1 | 2 | 3 | 4 | 5 | 6 | 7 | 8 |
| 1 | Glutathione S-transferase P (EC 2.5.1.18) (GST class-pi) (GSTP1-1) - | GSTP1_HUMAN | P09211 | 5 | 5 | 6 | 6 | 6 | 5 | 7 | 4 |
| | Homo sapiens (Human) | | | | | | | | | | |
| 2 | Transgelin-2 (SM22-alpha homolog) - Homo sapiens | TAGL2_HUMAN | P37802 | 7 | 5 | 6 | 5 | 5 | 6 | 5 | 9 |
| 3 | 6-phosphogluconate dehydrogenase, decarboxylating (EC 1.1.1.44) - | 6PGD_HUMAN | P52209 | 6 | 5 | 4 | 6 | 5 | 7 | 2 | 4 |
| | Homo sapiens (Human) | | | | | | | | | | |
| 4 | Vinculin (Metavinculin) - Homo sapiens (Human) | VINC_HUMAN | P18206 | 11 | 17 | 5 | 11 | 11 | 21 | 22 | 12 |
| 5 | ELAV-like protein 1 (Hu-antigen R) (HuR) - Homo sapiens | ELAV1_HUMAN | Q15717 | 2 | 2 | 1 | 1 | 4 | 2 | 2 | 2 |
| 6 | Transmembrane emp24 domain-containing protein 10 precursor | TMEDA_HUMAN | P49755 | 3 | 3 | 1 | 1 | 2 | 1 | | 2 |
| | (Transmembrane protein Tmp21) (21 kDa transmembrane-trafficking | | | | | | | | | | |
| | protein) (p24delta) (S31III125) (S31I125) (Tmp-21-I) - Homo sapiens | | | | | | | | | | |
| | (Human) | | | | | | | | | | |
| 7 | Galectin-3-binding protein precursor (Lectin galactoside-binding | LG3BP_HUMAN | Q08380 | 3 | 4 | 6 | 3 | 1 | | 2 | 1 |
| | soluble 3-binding protein) (Mac-2-binding protein) (Mac-2 BP) | | | | | | | | | | |
| | (MAC2BP) (Tumor-associated antigen 90K) - Homo sapiens (Human) | | | | | | | | | | |
| 8 | MARCKS-related protein (MARCKS-like protein 1) (Macrophage | MRP_HUMAN | P49006 | 1 | 2 | 2 | | 1 | 2 | | 1 |
| | myristoylated alanine-rich C kinase substrate) (Mac-MARCKS) | | | | | | | | | | |
| | (MacMARCKS) - Homo sapiens (Human) | | | | | | | | | | |
| 9 | Tenascin precursor (TN) (Tenascin-C) (TN-C) (Hexabrachion) | TENA_HUMAN | P24821 | | 9 | 1 | 3 | 6 | 2 | 21 | 6 |
| | (Cytotactin) (Neuronectin) (GMEM) (J1) (Myotendinous antigen) | | | | | | | | | | |
| | (Glioma-associated-extracellular matrix antigen) (GP 150-225) - Homo | | | | | | | | | | |
| | sapiens (Human) | | | | | | | | | | |
| 10 | 14-3-3 protein eta (Protein AS1) - Homo sapiens (Human) | 1433F_HUMAN | Q04917 | 2 | 1 | 2 | 1 | 2 | 1 | 2 | 2 |
| 11 | Proteasome activator complex subunit 1 (Proteasome activator 28-alpha | PSME1_HUMAN | Q06323 | 3 | 1 | 4 | 1 | 2 | 4 | 1 | 1 |
| | subunit) (PA28alpha) (PA28a) (Activator of multicatalytic protease | | | | | | | | | | |
| | subunit 1) (11S regulator complex subunit alpha) (REG-alpha) | | | | | | | | | | |
| | (Interferon gamma up-regulated I-5111 | | | | | | | | | | |
| 12 | Glucosidase 2 subunit beta precursor (Glucosidase II subunit beta) | GLU2B_HUMAN | P14314 | 3 | 4 | 5 | 2 | 3 | 6 | 5 | 4 |
| | (Protein kinase C substrate, 60.1 kDa protein, heavy chain) (PKCSH) | | | | | | | | | | |
| | (80K-H protein) - Homo sapiens | | | | | | | | | | |

| | Column | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|
| | 12 | 13 | 14 | 15 | 16 | 17 | 18 | 19 | 20 | 21 | 22 | 23 | 24 | 25 | 26 | 27 | 28 | 29 | 30 | 31 | 32 | 33 | 34 | 35 | 36 | 37 | 38 | 39 | 40 | 41 |
| | Sample# | | | | | | | | | | | | | | | | | | | | | | | | | | | | | |
| Row | 9 | 10 | 11 | 12 | 13 | 14 | 15 | 16 | 17 | 18 | 19 | 20 | 21 | 22 | 23 | 24 | 25 | 26 | 27 | 28 | 29 | 30 | 31 | 32 | 33 | 34 | 35 | 36 | 37 | 38 |
| 1 | 7 | 2 | 5 | 5 | 2 | 1 | 1 | 3 | 3 | 3 | 7 | 11 | 1 | 8 | 9 | 1 | 0 | 12 | 3 | 6 | 3 | 4 | 8 | 5 | 2 | 1 | 1 | 0 | 1 | 2 |
| 2 | 7 | 6 | 4 | 5 | 2 | 1 | | 2 | 3 | 3 | 6 | 6 | 1 | 6 | 7 | 4 | 2 | 5 | 0 | 6 | 5 | 1 | 1 | 6 | 0 | 1 | | 2 | 0 | 0 |
| 3 | 3 | 3 | 6 | 4 | 1 | 3 | 1 | 4 | 2 | 2 | 6 | 11 | 1 | 4 | 1 | 0 | 0 | 9 | 0 | 5 | 1 | 11 | 3 | 4 | 0 | | | 1 | 0 | 1 |
| 4 | 11 | 7 | 14 | 14 | 3 | 9 | 4 | 10 | 8 | 9 | 6 | 9 | | 4 | 16 | 1 | 0 | 4 | 1 | 3 | 1 | 2 | 0 | 4 | 0 | 4 | 1 | 0 | 0 | 2 |
| 5 | 1 | | 1 | 1 | 1 | 1 | 1 | 2 | 1 | 2 | 3 | 3 | 1 | 2 | 0 | 0 | 0 | 4 | 1 | 2 | 0 | 1 | 2 | 1 | 0 | 1 | 1 | 0 | 1 | 0 |
| 6 | 1 | 1 | 1 | 1 | | | | 1 | | | 1 | 2 | | 1 | 1 | 1 | 1 | 1 | 1 | 1 | 2 | 1 | 3 | 1 | 1 | 1 | | 0 | 0 | 0 |
| 7 | 1 | 5 | 2 | 1 | | 1 | | 1 | 1 | 2 | 2 | 5 | | 5 | 2 | 0 | 1 | 5 | 0 | 2 | 3 | 4 | 5 | 0 | 4 | 1 | 1 | 0 | 1 | 0 |
| 8 | 1 | 1 | 1 | 1 | 2 | 2 | 1 | 2 | 2 | 2 | 1 | 1 | 1 | 3 | 0 | 0 | 0 | 1 | 0 | 0 | 1 | 0 | 2 | 1 | 0 | | | 0 | 0 | 0 |
| 9 | 6 | | 1 | 4 | 2 | 3 | 2 | 5 | 1 | 1 | 1 | 2 | | 2 | 5 | 0 | 0 | 4 | 0 | 0 | 6 | 1 | 0 | 1 | 0 | | | 0 | 0 | 0 |
| 10 | 1 | 3 | 1 | 1 | 1 | 1 | 1 | 2 | 1 | 1 | 1 | 2 | 1 | 0 | 0 | 0 | 0 | 1 | 0 | 1 | 0 | 1 | 1 | 1 | 0 | | | 0 | 0 | 1 |
| 11 | 3 | 2 | 1 | 1 | | | | 4 | 1 | 2 | 1 | 3 | | 5 | 2 | 1 | 1 | 0 | 1 | 2 | 3 | 0 | 2 | 2 | 0 | | | 0 | 0 | 0 |
| 12 | 5 | 3 | 3 | 4 | | 2 | 1 | 3 | 2 | 1 | 2 | 3 | | 5 | 3 | 0 | 0 | 5 | 0 | 2 | 1 | 0 | 5 | 1 | 0 | | 1 | 0 | 1 | 1 |

| | Column | | | | | | | | | | | | | | |
|---|---|---|---|---|---|---|---|---|---|---|---|---|---|---|---|
| | | | | | | | | | | | | | | 55 | 56 |
| | 42 | 43 | 44 | 45 | 46 | 47 | 48 | | 50 | 54 | 52 | 53 | | % Cancers | % Normals |
| | Sample# | | | | | | | | # Peptides in | # Peptides in | Avg. Peptides per | Avg. Peptides per | 54 | Expressing | Expressing |
| Row | 39 | 40 | 41 | 42 | 43 | 44 | 45 | 49 | Cancer | Normal | Cancer Sample | Normal Sample | Ratio | Protein | Protein |
| 1 | 3 | 2 | 1 | 0 | 2 | | 0 | | 156 | 13 | 4.7 | 1.1 | 4.4 | 95% | 67% |
| 2 | 3 | 3 | 0 | 0 | 1 | | 0 | | 137 | 10 | 4.2 | 0.8 | 5.0 | 86% | 42% |
| 3 | 1 | 1 | 2 | 0 | 0 | | 0 | | 124 | 6 | 3.8 | 0.5 | 7.5 | 81% | 42% |
| 4 | 0 | 2 | 3 | 0 | 1 | | 0 | | 250 | 13 | 7.6 | 1.1 | 7.0 | 81% | 50% |
| 5 | 1 | 0 | 0 | 0 | 0 | | 0 | | 47 | 4 | 1.4 | 0.3 | 4.3 | 76% | 33% |
| 6 | 0 | 0 | 0 | 0 | 0 | | 0 | | 36 | 1 | 1.1 | 0.1 | 13.1 | 71% | 8% |
| 7 | 0 | 0 | 0 | 0 | 0 | | 0 | | 72 | 3 | 2.2 | 0.3 | 8.7 | 71% | 25% |
| 8 | 0 | 0 | 0 | 0 | 0 | | 0 | | 35 | 0 | 1.1 | 0.0 | NA | 67% | 0% |
| 9 | 0 | 0 | 0 | 0 | 0 | | 0 | | 95 | 0 | 2.9 | 0.0 | NA | 67% | 0% |
| 10 | 1 | 0 | 0 | 0 | 0 | | 0 | | 35 | 2 | 1.1 | 0.2 | 6.4 | 67% | 17% |
| 11 | 1 | 1 | 2 | 0 | 0 | | 0 | | 54 | 4 | 1.6 | 0.3 | 4.9 | 67% | 25% |
| 12 | 0 | 0 | 1 | 0 | 0 | | 1 | | 83 | 5 | 2.5 | 0.4 | 6.0 | 67% | 42% |

TABLE 3

| Protein | Biological Function | References |
|---|---|---|
| Glutathione | metabolic enzyme for detoxification of a | Chan et al. Clin Cancer Res. |
| S-transferase | wide range of electrophiles generated | 2005 Apr. 15; 11(8): 2981-5. |
| P (GSTP1-1) | through oxidative metabolism | Yokoyama et al. Gynecol |
| | | Oncol. 1998 March; 68(3): 280-7 |
| Transgelin-2 | Involved in actin cross-linking, calcium | Shaplan et al. J Cell Bio.l 1993 |
| | interactions, and contractile properties of | June: 1231(5): 1065-73 |
| | the cell | DeSouza et al. Mol Cell Prot. |
| | | 2007 Mar. 27 |
| 6-phosphogluconate | key regulatory enzyme of the pentose | Salvemini et al. J Biol. |
| dehydrogenase (6PGD) | phosphate pathway which produces | Chem. 1999 274(5): 2750-7 |
| | NADPH | Bonham et al. Br Med J. 1962 |
| | | 2(5308): 823-4 |
| Vinculin | Involved in cell adhesion. May be involved | Chen et al. J Biol. Chem. 2006 |
| (Metavinculin) | in the attachment of the actin-based | 281(52): 40389-98 |
| | microfilaments to the plasma membrane. | Carter et al. J. Cell Physiol. |
| | May also play important roles in cell | 1999 178(3): 320-32 |
| | morphology and locomotion. | |
| ELAV-like protein 1 | Key protein in the stability of certain | Ford et al. Genes Dev. 1999 |
| (HuR) | mRNA molecules and prevents RNA decay. | 13(2): 188-201 |
| | Expressed preferentially in growing cells. | Lopez et al. RNA Biol. 2005 |
| | | 2(1): 11-3 |
| Transmembrane emp24 | Vesicular trafficking protein that may direct | Baker et al. J. Urol. 2000 |
| (Tmp21) | the intracellular trafficking or secretion of | 164(2): 562-6 |
| | proteins responsible for nephrogenesis | |
| Galectin-3-binding | Glycolipid protein involved in cell adhesion | Elola et al. Cell Mol. Life Sci. |
| (Mac-2 binding | and growth regulation and thought to inhibit | 2007 (Eur ahead of pub) |
| protein) | cell adhesion to laminin matrices | Brastmann et al. Pathol Res |
| | | Pract. 2003 199(3): 151-8 |
| MARCKS-related | May be involved in coupling the protein | Ramsden J J. Int J. Biochem |
| protein | kinase C and calmodulin signal transduction | Cell Biol. 2000 32(5): 475-9 |
| | systems and may be essential to controlling | |
| | cell shape | |
| Tenascin | A glycan-binding protein with cell | Elola et al. Cell Mol. Life Sci. |
| | adhesion-modulating properties involved in | 2007 (Eur ahead of pub) |
| | cell adhesion, spreading, and migration | Sedele et al. Int. J Gynecol. |
| | | Pathol. 2002 21(2): 161-6 |
| | | Sasono et al. Mod Pathol. 1993 |
| | | 6(3): 323-6 |
| 14-3-3 protein eta | Member of a family | Tzivion et al. Semin Cancer |
| | phosphoserine/threonine binding proteins | Biol. 2006 16(3): 203-13 |
| | that play critical roles in cell signaling | Yaffe M B. FEBS Lett. 2002 |
| | events that control the cell cycle, | 513(1):53-7 |
| | transcriptional alterations in response to | |
| | environmental cues, and programmed cell | |
| | death | |
| Proteasome activator | Involved in activating the intracellular | Song et al. J Biol Chem. 1997 |
| (PA28alpha) | proteasome for non-lysosomal protein | 272(44): 27994-8000 |
| | degradation | |
| Glucosidase 2 beta | GBA2 is a glucosylceramidase, a protein | Boot et al. J Biol Chem. 2007 |
| | involved in the metabolism of bile acid- | 282(2): 1305-12 |
| | glucose conjugates, and when no | |
| | functioning causes accumulation of | |
| | glycolipids and the endoplasmic reticulum | |
| | storage disease, Gaucher's disease | |

An additional description of this experiment is found in the poster presentation of Krizman et al., “Discovery of Novel Protein Biomarkers in Formalin Fixed Paraffin Embedded Endometrial Cancer Tissue by Mass Spectrometry,” presented at the American Association for Cancer Research (AACR) annual meeting on Tuesday, Apr. 15, 2008, which is incorporated herein in its entirety.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

All publications identified above or in the attachments hereto are incorporated herein by reference in their entirety.

1. A method of diagnosing, prognosing, and determining the therapeutic target of endometrial cancer in a human patient comprising the steps of:
a) detecting the presence and level of expression of one, two or more of the proteins of Tables 1 and 2 in a sample from a human patient, said sample comprising endometrial tissue, endometrial cells, or a bodily fluid containing proteins from said patient's endometrium; and
b) comparing the level of expression of said one or more proteins to the level of expression of said one, two, or more proteins in normal endometrial tissue, endometrial cells, or bodily fluid containing proteins from said normal endometrium, wherein differential expression of said one, two, or more proteins indicates the presence of endometrial cancer in said patient.

2. The method of claim 1, wherein said differential expression is overexpression, underexpression, or some combination of both for all or some of said one, two, or more proteins in said sample.

3-21. (canceled)

22. The method of claim 1, wherein said endometrial tissue consists essentially of endometrial epithelial cells.

23. The method of claim 1, wherein said bodily fluids are fractionated or unfractionated blood, serum, plasma, or vaginal secretions, and cytology specimens.

24. The method of claim 1, wherein the tissue is a biopsy sample or a sample of a tissue obtained surgically.

25. The method of claim 24, wherein the tissue is fresh, fresh frozen, or chemically fixed and preserved.

26. The method of claim 25, wherein said chemical fixation and preservation comprises formalin fixation and embedding in paraffin.

27-28. (canceled)

29. The method of claim 1, wherein said proteins are measured as intact, full-length proteins or are measured by measuring multiple or individual peptides derived from said proteins.

30. The method of anyone of claim 1 or claim 29, wherein said proteins are detected and measured by mass spectroscopy.

31. The method of claim 30, wherein said proteins are detected and assayed by an SRM or MRM assay utilizing a labeled synthetic peptide as a quantitative standard against which to compare the amount of a specific peptide and protein, or peptides and proteins.

32. The method of claim 30, wherein said proteins are detected and assayed by an SRM or MRM assay not utilizing a labeled synthetic peptide as a quantitative standard against which to compare the amount of a specific peptide and protein, or peptides and proteins.

33. (canceled)

34. The method of claim 30, wherein said mass spectroscopy is selected from the group consisting of LC-ESI-MS/MS, MALDI-MS, tandem MS, TOF/TOF, TOF-MS, TOF-MS/MS, triple-quad MS, and triple-quad MS/MS.

35. (canceled)

36. The method of claim 1 or 2, wherein said proteins are detected and their levels of expression are determined by a protein microarray or by an immunoassay.

37. The method of claim 1 or claim 30, wherein said immunoassay is selected from the group consisting of immunohistochemistry, Western blot, dot blot, and ELISA.

38-159. (canceled)

160. A kit for the detection of endometrial disease in a human patient comprising an antibody to one, two, or more of the proteins of Tables 1 and 2, or an antibody to one or more peptides derived by fragmentation of said one, two, or more proteins of Tables 1 and 2.
